(19) United States
(12) Patent Application Publication
(10) Pub. No.: US 2025/0120771 A1
(43) Pub. Date: Apr. 17, 2025

(54) SYSTEMS AND METHODS FOR PLANNING JOINT ALIGNMENT IN ORTHOPEDIC PROCEDURES

(71) Applicant: MicroPort Orthopedics Holdings Inc., Arlington, TN (US)

(72) Inventors: Jake Adams, Conroe, TX (US); Jinling Sun, Mansfield, MA (US); Brian R. Harris, JR., Cordova, TN (US); Paul S. Bryant, Collierville, TN (US); C. Brian McDaniel, Arlington, TN (US); Fred W. Bowman, Germantown, TN (US)

(73) Assignee: MicroPort Orthopedics Holdings Inc., Arlington, TN (US)

(21) Appl. No.: 18/907,996

(22) Filed: Oct. 7, 2024

Related U.S. Application Data
(60) Provisional application No. 63/589,544, filed on Oct. 11, 2023.

(51) Int. Cl. A61B 34/20; G06T 7/00

(52) U.S. Cl. CPC A61B 34/20; G06T 7/0012; G06T 2207/20081; G06T 2207/30008

(57) ABSTRACT

Systems, apparatuses, and methods concerning identifying and classifying target orthopedic joints using one or more deep learning networks and alignment angles, wherein the deep learning networks are configured to identify one or more alignment angles of the identified target orthopedic joint to then classify the target orthopedic joint into one or more classes selected from a set of pre-defined classes. Exemplary systems, apparatuses, and methods may further comprise recommending a type of surgical procedure based on the classification of the identified target orthopedic joint.

### RELATED APPLICATION

This application claims the benefit of U.S. Provisional Application No. 63/589,544 filed on Oct. 11, 2023. The disclosure of this related application is hereby incorporated into the present disclosure in its entirety.

### BACKGROUND OF THE INVENTION

### 1. Technical Field

The present disclosure relates generally to the field of orthopedic surgery, and more particularly to systems, apparatuses, and methods that augment data visualization and surgical recommendations based on data derived from individual patients.

### 2. Related Art

An emerging objective of orthopedic joint replacement surgeries is to balance the competing interests of restoring the natural alignment and the rotational axis or axes of the pre-diseased joint with orienting the endoprosthetic implant in a mechanically stable manner to prolong the implant's useful life. However, these objectives can be difficult to achieve in practice because restoring natural alignment has traditionally been seen to come at the expense of some implants' mechanical stability, while orienting the implant in a mechanically stable manner has traditionally been seen to come at the expense of patient comfort.

Furthermore, common joint diseases are degenerative diseases. Osteoarthritis is one such example. Commonly occurring in the hands, hips, or knees, osteoarthritis is characterized by the wearing down of the cartilage between articulating bones. When cartilage is absent, the adjacent articulating bones begin to wear down and change shape. With the change in joint structure, the axis or axes of articulation likewise change away from the pre-diseased or “constitutional” axis or axes.

Without radiographic images of the pre-diseased joint, or without sufficient remaining hyaline cartilage adjacent in the joint that can be used to estimate the amount of pre-diseased hyaline cartilage, it is difficult to reconstruct the natural alignment of the patient's pre-diseased joint with certainty.

This has led some surgeons to abandon restorative alignment altogether. Other surgeons opt to approximate the constitutional joint line based on pre- or intraoperative measurements of the patient's specific anatomy in favor of patient comfort; however, placement of the endoprosthetic implant in a manner that approximates the pre-diseased joint line, may place the implant at an angle and subject the implant to uneven mechanical forces over time, which may shorten the useful life of the implant.

### SUMMARY OF THE INVENTION

The problems of the prior art can be solved by an orthopedic image processing system comprising: an input data set, the input data set comprising at least one tissue-penetrating image of a target orthopedic joint, one or more processors, and memory storing instructions that, when executed by the one or more processors, cause the one or more processors to perform operations, the operations comprising: identifying at least two bones comprising a target orthopedic joint to define an identified orthopedic joint; identifying an area of bone or soft tissue loss in the identified orthopedic joint to define an identified loss area; applying an adjustment algorithm to replace the identified loss area with a reconstructed area to thereby define a reconstructed orthopedic joint; and identifying an alignment angle of the reconstructed orthopedic joint to define a reconstructed alignment angle.

In certain exemplary embodiments, the orthopedic image processing system can be further configured to return a predicted constitutional joint line based on the reconstructed alignment angle.

In certain exemplary embodiments, the orthopedic image processing system can be further configured to identify multiple reconstructed alignment angles.

In certain exemplary embodiments, the orthopedic image processing system can be further configured to classify the reconstructed constitutional joint based on a value of the reconstructed alignment angle.

In yet further exemplary embodiments, the processing system can display a recommended surgical alignment procedure based on the value of the reconstructed alignment angle or based on the classification of the reconstructed orthopedic joint.

In still yet further exemplary embodiments, the processing system can display a recommended implant type based on the value of the reconstructed alignment angle or based on the classification of the reconstructed joint.

In yet another exemplary embodiment, a size of an implant or of a trial implant may be recommended by the processing system based on the identified orthopedic joint, or substructures thereof.

### BRIEF DESCRIPTION OF THE DRAWINGS

The foregoing will be apparent from the following more particular description of exemplary embodiments of the disclosure, as illustrated in the accompanying drawings. The drawings are not necessarily to scale, with emphasis instead being placed upon illustrating the disclosed embodiments.

FIG. 1 is a schematic representation of an exemplary orthopedic image processing system comprising an input data set comprising at least one tissue-penetrating image of a target orthopedic joint, and a processor configured to perform operations, the operations comprising: identifying at least two bones comprising a target orthopedic joint, reconstructing the anatomy of a pre-diseased orthopedic joint, and identifying one or more alignment angles of the reconstructed orthopedic joint.

FIG. 2 is a schematic representation of an exemplary orthopedic image processing system further comprising different types of input data, in which a processor performs operations, the operations comprising: identifying the target orthopedic joint, reconstructing the anatomy of a pre-diseased orthopedic joint, and identifying one or more alignment angles of the reconstructed orthopedic joint, wherein the system further comprises a display for displaying processor outputs, and a surgical robotic arm configured to adjust the position of a tool end of the surgical robotic arm based on one or more outputs from the processor.

FIG. 3A is a simplified representation of an anterior view of an identified target orthopedic joint, in which the identified orthopedic joint is a knee experiencing the degenerative effects of osteoarthritis on the distal medial femoral condyle. Identified alignment angles of the diseased joint are shown for reference and include the lateral distal femoral angle (“LDFA”), the medial proximal tibia angle (“MPTA”), and the mechanical hip-knee-ankle (“mHKA”) angle.

FIG. 3B is a simplified representation of an anterior view of a reconstructed orthopedic joint, in which the reconstructed orthopedic joint is a knee, and wherein the identified alignment angles include the lateral distal femoral angle (“LDFA”), the medial proximal tibia angle (“MPTA”), and the mechanical hip-knee-ankle (“mHKA”) angle of the reconstructed orthopedic joint.

FIG. 4 is a schematic representation of a convolutional neural network, a type of exemplary deep learning network that can be used with systems and methods in accordance with this disclosure.

FIG. 5 is a schematic representation of a computer platform that can be configured to perform any of the exemplary methods described herein, or be used with any of the exemplary systems disclosed herein.

FIG. 6 is a schematic representation of an exemplary convolutional neural network configured to identify an alignment angle of the identified orthopedic joint or of a reconstructed orthopedic joint.

FIG. 7 is a schematic representation of an exemplary U-net convolutional neural network that is further configured to identify a target orthopedic joint and an alignment angle of the identified or of a reconstructed orthopedic joint.

FIG. 8A represents a sample 2D input data set sourced from a tissue-penetrating image off the target orthopedic joint.

FIGS. 8B and 8C each depict a bone of the target orthopedic element, in which a mask has been applied over one of the target bones to collectively illustrate how a processor can define or output an identified orthopedic joint.

FIG. 9 is another schematic representation of an exemplary U-net convolutional neural network configured to identify the orthopedic element and further configured to identify key anatomical points on the target or reconstructed orthopedic element for the purposes of identifying alignment angles on the target or reconstructed orthopedic element.

FIG. 10 is a representation of a 2D input data set from a tissue-penetrating image of the target orthopedic joint in which key anatomical markers have been identified using the model of FIG. 9.

FIG. 11 is a schematic representation of a system configured to identify an orthopedic element and to align components of endoprosthetic implant components using two or more tissue-penetrating, flattened, input images taken of the same subject orthopedic element from calibrated detectors at an offset angle.

FIG. 12 is a schematic depiction of a pinhole camera model used to convey how principles of epipolar geometry can be used to ascertain the position of a point in 3D space from two 2D images taken from different reference frames from a calibrated image detector.

FIG. 13 is a flow chart illustrating steps of an exemplary method.

FIG. 14 represents an exemplary embodiment of an orthopedic image classification system

FIG. 15A is an image of subject orthopedic elements taken from the anterior-posterior (“A-P”) position that shows an exemplary calibration jig.

FIG. 15B is an image of subject orthopedic elements of FIG. 15A taken from the medial-lateral (“M-L”) position that shows an exemplary calibration jig.

FIG. 16A is a schematic representation of a 3D input data set taken from a tissue-penetrating image of the target orthopedic joint.

FIG. 16B is a representation of the training set for a deep learning model configured to identify the target orthopedic joint and an alignment angle from the 3D input data set of FIG. 16A.

FIG. 16C is a representation of the mask training set for a deep learning model configured to identify the target orthopedic joint and an alignment angle from the 3D input data set of FIG. 16A.

FIG. 16D is a representation of the output of the deep learning model configured to identify the target orthopedic joint and an alignment angle from the 3D input data set of FIG. 16A.

FIG. 16E illustrates how exemplary lines can be ascertained from two points identified using the exemplary deep learning model, which in turn can be used to calculate alignment angles, which may then in turn be used to classify the identified orthopedic joint.

### BRIEF DESCRIPTION OF THE PREFERRED EMBODIMENTS

The following detailed description of the preferred embodiments is presented only for illustrative and descriptive purposes and is not intended to be exhaustive or to limit the scope and spirit of the invention. The embodiments were selected and described to best explain the principles of the invention and its practical application. One of ordinary skill in the art will recognize that many variations can be made to the invention disclosed in this specification without departing from the scope and spirit of the invention.

Similar reference characters indicate corresponding parts throughout the several views unless otherwise stated. Although the drawings represent embodiments of various features and components according to the present disclosure, the drawings are not necessarily to scale and certain features may be exaggerated to better illustrate embodiments of the present disclosure, and such exemplifications are not to be construed as limiting the scope of the present disclosure.

Except as otherwise expressly stated herein, the following rules of interpretation apply to this specification: (a) all words used herein shall be construed to be of such gender or number (singular or plural) as such circumstances require; (b) the singular terms “a,” “an,” and “the,” as used in the specification and the appended claims include plural references unless the context clearly dictates otherwise; (c) the antecedent term “about” applied to a recited range or value denotes an approximation with the deviation in the range or values known or expected in the art from the measurements; (d) the words, “herein,” “hereby,” “hereto,” “hereinbefore,” and “hereinafter,” and words of similar import, refer to this specification in its entirety and not to any particular paragraph, claim, or other subdivision, unless otherwise specified; (c) descriptive headings are for convenience only and shall not control or affect the meaning of construction of part of the specification; and (f) “or” and “any” are not exclusive and “include” and “including” are not limiting. Further, the terms, “comprising,” “having,” “including,” and “containing” are to be construed as open-ended terms (i.e., meaning “including but not limited to”).

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether explicitly described.

To the extent necessary to provide descriptive support, the subject matter and/or text of the appended claims are incorporated herein by reference in their entirety.

Recitation of ranges of values herein are merely intended to serve as a shorthand method of referring individually to each separate value falling within the range of any sub-ranges there between, unless otherwise clearly indicated herein. Each separate value within a recited range is incorporated into the specification or claims as if each separate value were individually recited herein. Where a specific range of values is provided, it is understood that each intervening value, to the tenth or less of the unit of the lower limit between the upper and lower limit of that range and any other stated or intervening value in that stated range of sub range thereof, is included herein unless the context clearly dictates otherwise. All subranges are also included. The upper and lower limits of these smaller ranges are also included therein, subject to any specifically and expressly excluded limit in the stated range.

The terms, “horizontal” and “vertical” are used to indicate direction relative to an absolute reference, i.e., ground level. However, these terms should not be construed to require structure to be absolutely parallel or absolutely perpendicular to each other. For example, a first vertical structure and a second vertical structure are not necessarily parallel to each other.

Throughout this disclosure and unless otherwise noted, various positional terms, such as “distal,” “proximal,” “medial,” “lateral,” “anterior,” and “posterior,” will be used in the customary manner when referring to the human anatomy. More specifically, “distal” refers to the area away from the point of attachment to the body, while “proximal” refers to the area near the point of attachment to the body. For example, the distal femur refers to the portion of the femur near the tibia, whereas the proximal femur refers to the portion of the femur near the hip. The terms, “medial” and “lateral” are also essentially opposites. “Medial” refers to something that is disposed closer to the middle of the body. “Lateral” means that something is disposed closer to the right side or the left side of the body than to the middle of the body. Regarding, “anterior” and “posterior,” “anterior” refers to something disposed closer to the front of the body, whereas “posterior” refers to something disposed closer to the rear of the body.”

“Varus” and “valgus” are broad terms and include without limitation, rotational movement in a medial and/or lateral direction relative to the knee joint.

The term, “mechanical axis” of the femur refers to an imaginary line drawn from the center of the femoral head to the center of the distal femur at the knee.

The term, “anatomic axis” refers to an imaginary line drawn lengthwise down the middle of femoral shaft or tibial shaft, depending upon use.

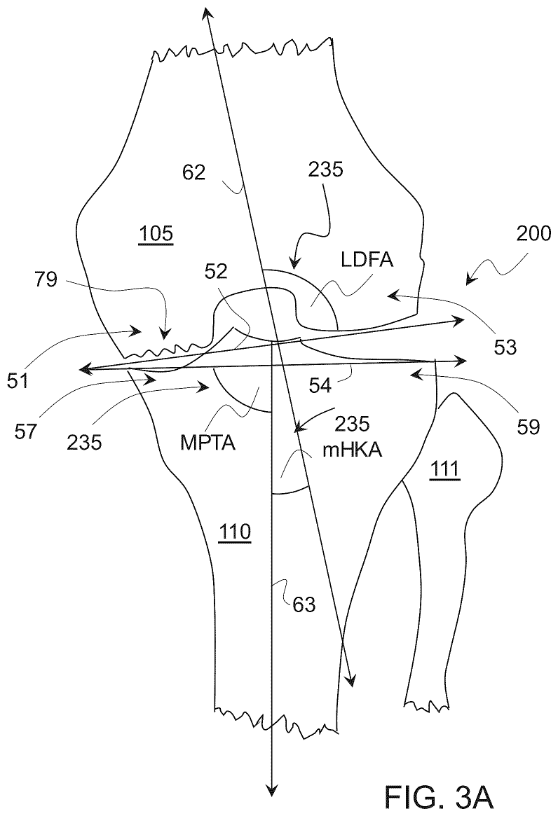

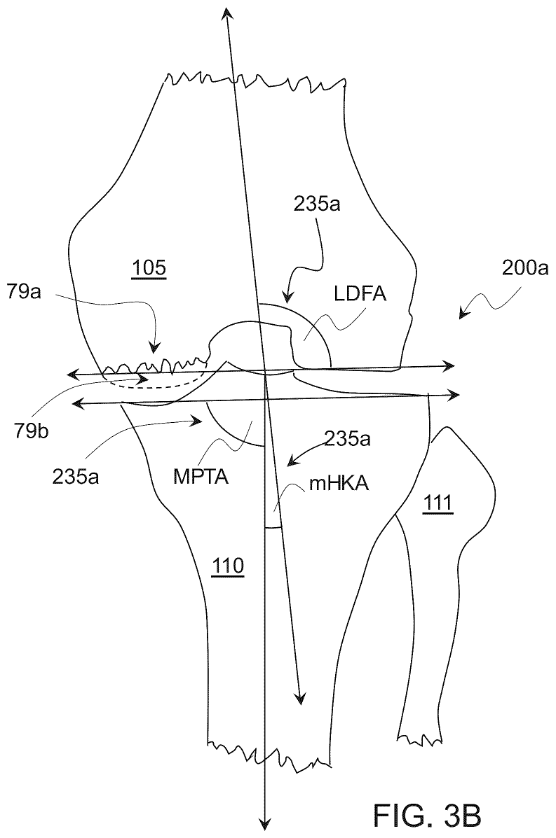

To illustrate the principles and detailed elements of embodiments in accordance with this disclosure, a detailed description of exemplary embodiments used with a knee joint will be described herein (see FIGS. 3A and 3B). However, it will be appreciated that the exemplary methods and systems described herein can be applied to a variety of orthopedic joints. It will be understood that the “target orthopedic joint” 200 referenced throughout this disclosure is not limited to the anatomy of a knee joint, but can include any other orthopedic joint in a vertebrate animal, including but not limited to humans. By way of example, such target orthopedic joints can include but are not limited to hip, shoulder, elbow, ankle, wrist, intercarpal, metatarsophalangeal, and interphalangeal joints.

Briefly, in a primary total knee arthroplasty (“TKA”), the surgeon typically makes a vertical medial parapatellar incision of about five to six inches in length on the anterior or anteromedial aspect of the knee. The surgeon then continues to incise the fatty tissue to expose the anterior or anteromedial aspect of the joint capsule. The surgeon may then perform a medial parapatellar arthrotomy to pierce the joint capsule. A retractor may then be used to move the patella generally laterally (roughly about 90 degrees) to expose the distal condyles of the femur and the cartilaginous meniscus resting on the proximal tibial plateau. The surgeon then removes the meniscus and uses instrumentation to measure and resect the distal femur and proximal tibia to accommodate trial implants.

Ultimately, a final endoprosthetic implant will be selected and assembled based on the sizing and the movement mechanics of the trial implants. The final implant typically comprises a femoral component that is placed on the resected distal femur, a tibial component that is placed on the resected proximal tibia, and an insert (typically known as a “tibial insert,” “a poly,” or a “meniscal insert”) that is disposed between the implanted femoral component and the implanted tibial component.

The placement of these final implant components, and by extension, the location of the resected surfaces upon which the respective implant components are engaged, largely dictates the position of the reconstructed joint line. In a typical case, a patient's natural pre-diseased, or “constitutional” joint line is not known prior to surgery. Various tools and methods can be used to estimate the constitutional joint line based upon the surviving intraoperative anatomy, but because many diseases that predicate a TKA are degenerative, it is difficult to ascertain the location of the constitutional joint line with certainty based upon intraoperative measurements that are obtained from worn or otherwise degenerated anatomy. This problem is especially pronounced in patients who experience arthritic bone loss at the central condyle contact points.

FIG. 3A illustrates this principle. FIG. 3A is an anterior view of a simplified representation of the boney anatomy of a target orthopedic joint 200. In the depicted example, the target orthopedic joint 200 is a knee joint. The knee comprises a distal aspect of the femur 105 disposed over the proximal aspect of the tibia 110. The proximal fibula 111 is shown adjacent to the proximal tibia 110. Various soft tissues connect the depicted bones (and the patella, not depicted here). The soft tissues can include ligaments, tendons, muscles, cartilage, skin, and any other non-boney anatomy of the target orthopedic joint 200.

The distal femur 105 comprises a medial distal femoral condyle 51 laterally disposed from a lateral distal femoral condyle 53. Likewise, the proximal tibia 110 comprises a medial tibial hemicondyle 57 that is laterally disposed from a lateral tibial hemicondyle 59. In a healthy knee (see FIG. 3B), the respective distal femoral condyles 51, 53 are disposed over the respective proximal tibial hemicondyles 57, 59 and a layer of hyaline articular cartilage is disposed between the respective condyles 51, 53 and hemicondyles 57, 59.

FIGS. 3A and 3B further depict several alignment angles 235 superimposed on the target orthopedic joint 200. The values of these alignment angles 235 can be used to classify the target orthopedic joint 200. In FIGS. 3A and 3B, the alignment angles 235 comprise the mechanical lateral distal femoral angle (“LDFA”), the mechanical medial proximal tibial angle (“MPTA”), and the mechanical hip knee ankle (“mHKA”) angle.

The LDFA is the lateral angle formed at the intersection of the distal femur joint line 52 and the femoral mechanical axis 62. The MPTA is the medial angle formed at the intersection of the proximal tibia joint line 54 and the tibial mechanical axis 63. The mHKA angle is the acute angle formed by the intersection of the femoral mechanical axis 62 and the tibial mechanical axis 63.

FIG. 3A represents a knee that suffers from a degenerative disease such as osteoarthritis. The disease progression is represented by an area of bone and soft tissue loss 79 on the articular surface of the medial femoral condyle 51. In knees that suffer from osteoarthritis, the hyaline articular cartilage that is normally disposed between the distal femur 105 and the proximal tibia 110 can be worn away in places. In advanced cases, the underlying bone (see 79) can also be worn away due to disease progression and repeated flexion and extension of the knee. FIG. 3A illustrates that a change in the underlying physical anatomy of a joint changes the alignment of the joint (e.g., compare the alignment angles 235 of FIG. 3A to those of FIG. 3B). This change in alignment often contributes to patient discomfort. Moreover, the uneven load distribution resulting from this degenerative alignment change can hasten further joint deterioration if surgical intervention is avoided.

A challenge that surgeons face in such advanced cases is determining precisely how much alignment-affecting bone or soft tissue has been lost. In the absence of detailed tissue-penetrating images of the patient's healthy knee, surgeons who are interested in restoring the patient's pre-diseased alignment generally estimate the amount of bone or soft tissue loss 79 based on intraoperative measurements or based on a simple subtraction of the LDFA from the MPTA. These methods cannot guarantee the restoration of the patient's pre-diseased joint line with accuracy and precision because these methods rely heavily on the state of the patient's already deteriorated orthopedic joint 200.

Moreover, many different surgical alignment procedures can exist for the same type of target orthopedic joint 200. The orientation of a surgically implanted endoprosthetic implant in a desired location to achieve patient comfort and prolong the implant's useful life depends in part upon the type of surgical alignment procedure that the surgeon elects. The lack of accurate and precise measurements of the alignment angles 235 of a diseased joint compared to accurate and precise alignment angles 235 of a reconstructed joint previously prevented surgeons from making completely informed decisions about the surgical alignment procedure and implant placement that would be most likely to benefit the surgeon's patient.

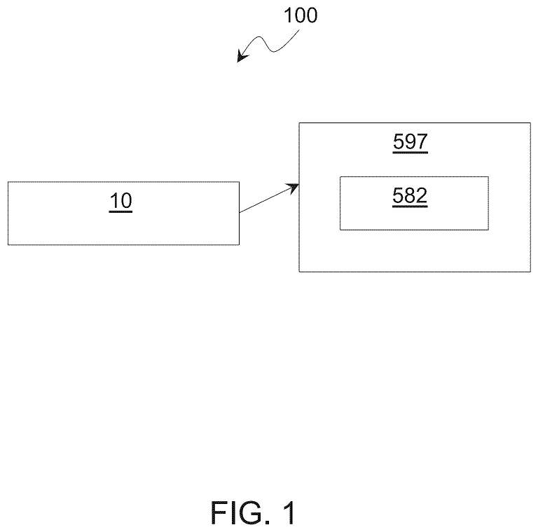

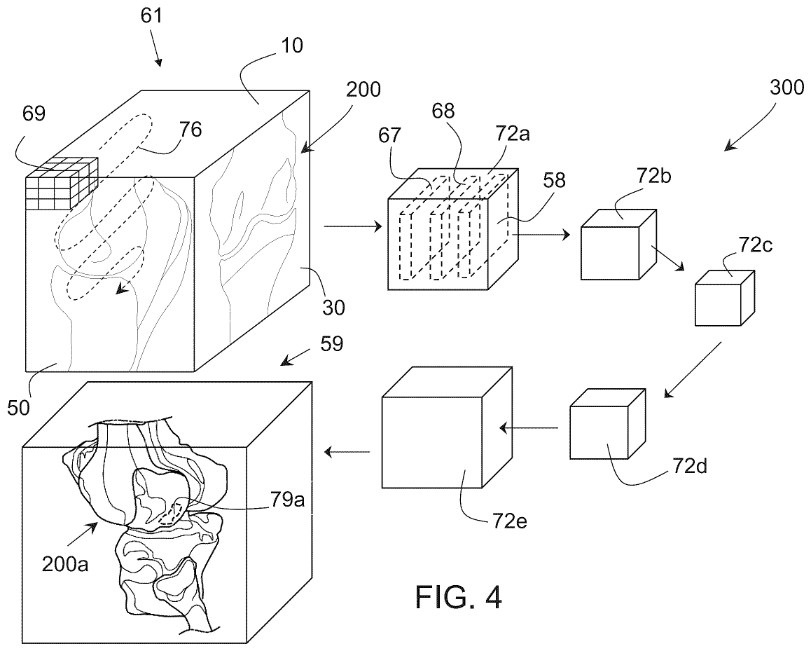

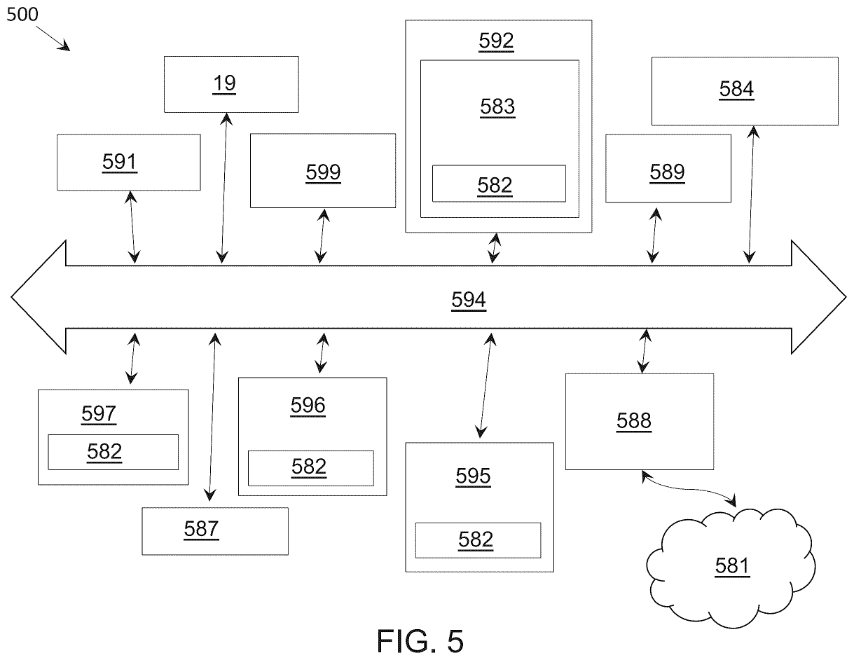

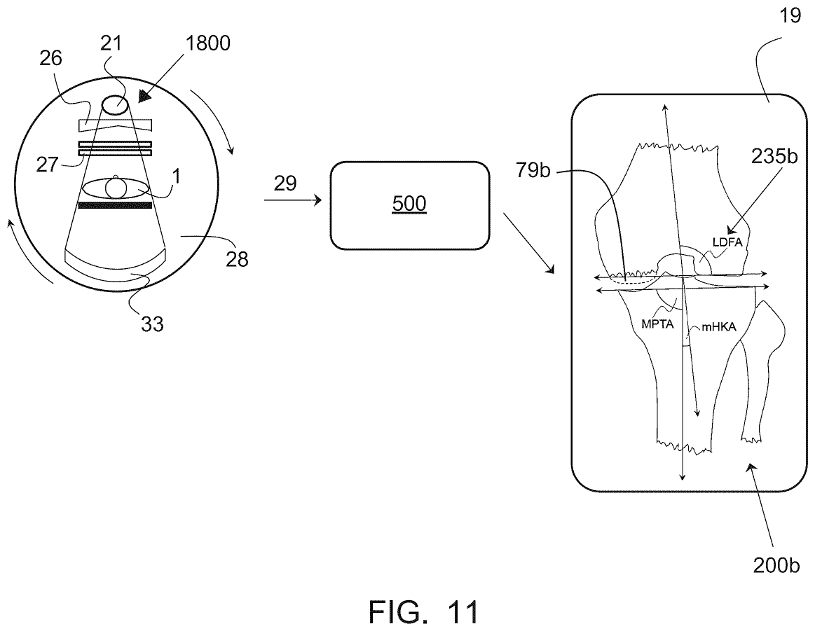

To address these problems, exemplary embodiments in accordance with this disclosure are provided. FIG. 1 is a schematic representation of an exemplary orthopedic image processing system 100 comprising: an input data set 10, the input data set 10 comprising at least one tissue-penetrating image of a target orthopedic joint 200 (FIG. 3A), a processor 597, and memory storing instructions 582 that, when executed by the processor 597, cause the processor 597 to perform operations: the operations include identifying the target orthopedic joint 200, identifying areas of bone or soft tissue loss 79, applying an adjustment algorithm to replace the identified loss area 79a with a reconstructed area 79b to thereby define a reconstructed constitutional joint 200b; and identify an alignment angle 235 of the identified joint 200a or of the reconstructed orthopedic joint 200b to define an identified alignment angle 235a or a reconstructed alignment angle 235b respectively (see LDFA, MPTA, mHKA in FIG. 3B). One or more of these operations may be performed with the aid of one or more deep learning networks (300 see FIGS. 4, 6, 7 and 9). That is, the one or more deep learning networks 300 can be configured to identify the target orthopedic joint 200 to define an identified orthopedic joint 200a, wherein the one or more deep learning networks 300 can be further configured to identify areas of bone or soft tissue loss 79 in the identified orthopedic joint 200a to define an identified loss area 79a (FIG. 4), wherein the one or more deep learning networks 300 can be configured to apply an adjustment algorithm to replace the identified loss area 79a with a reconstructed area 79b to thereby define a reconstructed constitutional joint 200b (FIG. 3B, FIG. 11), and wherein the one or more deep learning networks 300 can be further configured to identify one or more alignment angles 235 of the reconstructed orthopedic joint 200b to define one or more identified alignment angles 235a or one or more reconstructed alignment angles 235b. In certain exemplary embodiments, the processing system 100 can further be configured to return a predicted constitutional joint line based on the reconstructed alignment angle 235b. In other certain exemplary embodiments, the processor 597 can be further configured to categorize the identified orthopedic joint 200a or the reconstructed constitutional joint 200b into one or more anatomical categories based on one or more values of one or more identified alignment angles 235a or one or more reconstructed alignment angles 235b. The processor 597 can be part of a computer platform 500 (FIG. 5).

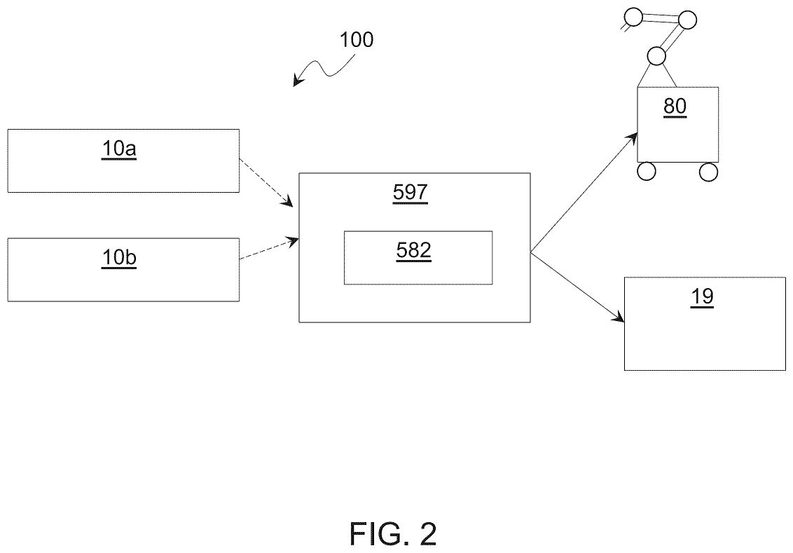

FIG. 2 is a schematic representation of an exemplary orthopedic image processing system 100 described above with reference to FIG. 1. FIG. 2 further illustrates that outputs from the processor 597 can be transmitted to a display 19 for viewing. The display 19 may optionally display any of the items identified by the exemplary systems and methods described herein, including but not limited to the identified orthopedic joint 200a, the one or more identified alignment angles 235a, a predicted constitutional joint line, a classification of the identified orthopedic joint 200a, a reconstructed orthopedic joint 100b, a classified orthopedic joint 200c, a reconstructed alignment angle 235b, an implant, a trial implant, a surgical instrument, subcomponents of any of the implant, trial implant, or surgical instrument, the position of the implant, trial implant, surgical instrument, or subcomponents thereof relative to the identified or reconstructed joint 200a, 200b, or a recommended surgical alignment procedure based upon the classification of the identified orthopedic joint 200a. All combinations and permutations of the foregoing are considered to be within the scope of this disclosure.

This display 19 may take the form of a screen. In other exemplary embodiments, the display 19 may comprise a glass or plastic surface that is worn or held by the surgeon or other people in the operation theater. Such a display 19 may comprise part of an augmented reality device, such that the display 19 shows the 3D model in addition to the wearer's visual field. In certain embodiments, such a 3D model can be superimposed on the actual identified orthopedic joint 200a. In yet other exemplary embodiments, the 3D model can be “locked” to one or more features of the identified orthopedic joint 200a, thereby maintaining a virtual position of the 3D model relative to the one or more features of the identified orthopedic joint 200a independent of movement of the display 19. It is still further contemplated that the display 19 may comprise part of a virtual reality system in which the entirety of the visual field is simulated.

In exemplary embodiments, it is contemplated that an identified component of an endoprosthetic implant, or the representative model of the component of the endoprosthetic implant can be superimposed on the bone of the identified orthopedic joint 200a into which the component of the endoprosthetic implant will be seated (e.g., the femoral component and the distal femur 105; the tibial component and the proximal tibia 110, etc.). The superimposition can be calculated and displayed using the mapped spatial data 43 of the respective identified elements.

In this manner, the surgeon and others in the operating room can have a near real time visualization of the component of the endoprosthetic implant and the identified orthopedic joint 200a in three dimensions and their alignment relative to one another.

Furthermore, because spatial data 43 of an identified component of an endoprosthetic implant and because spatial data 43 of the identified orthopedic joint 200a can be obtained from exemplary systems described herein, the degree of alignment can be calculated and further displayed on a display 19 in exemplary system embodiments. By way of example, the display 19 may optionally display a “best fit” percentage in which a percentage reaching or close to 100% reflects the alignment of an identified component of an endoprosthetic implant (e.g., a femoral component) relative to the reference distal femur 105 of the identified orthopedic joint 200a.

In embodiments in which the identified component of an endoprosthetic implant is a femoral component of a knee implant and in which the identified orthopedic joint 200a is a knee joint that comprises a distal femur 105 into which the femoral component will be inserted and seated, exemplary systems may display the varus or valgus angle of the longitudinal axis of the femoral component relative to the anatomical axis of the femur (i.e., the central axis of the femur extending through the intramedullary canal of the femur) or relative to the mHKA.

In certain embodiments, the output from the processor 597 can be transmitted to a surgical robot 80 to inform the surgical robot 80 to position a medical device, such as an implant, trial implant, instrument, or subcomponents of any of the foregoing, to track or match the position of a corresponding virtual medical device, such as an implant, trial implant, instrument, or subcomponents of any of the foregoing, displayed on a display 19 in an exemplary system in accordance with this disclosure.

FIG. 2. further illustrates that the input data set 10 can come from at least one of a variety of types of tissue-penetrating images. For example, tissue-penetrating images can be produced via flat panel X-ray radiography, magnetic resonance imaging (“MRI”), or computed tomography (“CT”) imaging. In the depicted embodiment, input data set 10a represents input data having two spatial dimensions (“2D”) Input data set 10b represents input data having three spatial dimensions (i.e., “3D”), which may include data derived from CT scans, MRI scans, or derived from reconstructive radiographic photogrammetry, such as for example, by processes or systems disclosed in U.S. patent application Ser. No. 17/835,894, the entirety of which is incorporated herein by reference.

One or more deep learning networks (also known as a “deep neural network” (“DNN”), such as a convolutional neural network (“CNN”), recurrent neural network (“RNN”), modular neural network, or sequence to sequence model, can be used to identify the target orthopedic joint 200 from the input data set 10.

### Photogrammetry Embodiments

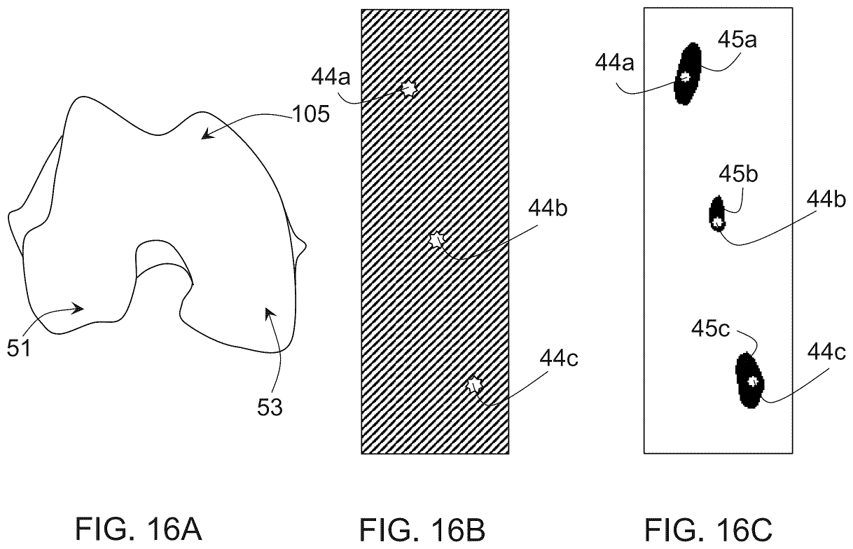

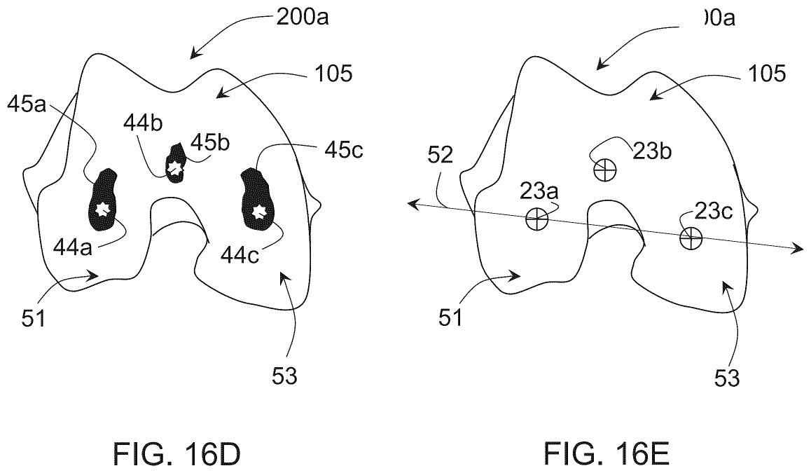

All the figures, but particularly FIGS. 4, and 11-13, and 15a-15b can be used to illustrate aspects of exemplary embodiments of the present disclosure in which at least two 2D radiographic input images of the same target orthopedic joint 200 taken at different angles, may be used with epipolar geometry to reconstruct a 3D volume of the imaged area, identify the target orthopedic joint 200, calculate the value of one or more alignment angles 235, and optionally categorize the identified orthopedic joint 200a based on a value of the one or more alignment angles 235. Other exemplary embodiments may use 3D input images (e.g., CT scans, MRI scans, etc.) in lieu of or in addition to 2D tissue-penetrating input images (see FIGS. 16A-16E). It will be appreciated that in embodiments comprising the use of 3D input tissue-penetrating images, the below-described photogrammetry steps can be optional.

In recent years, it has become possible to use 2D tissue-penetrating images, such as X-ray radiographs, to create 3D models of an imaged area. These models can be used preoperatively to plan surgeries much closer to the date of the actual surgery. These models can also be used intraoperatively (e.g., when projected on a display 19 or across a surgeon's field of view). Additionally, more providers may have access to flat panel X-ray radiography machines compared to providers who may have access to more complicated and expensive MRI or CT imaging machines.

However, traditional X-ray radiographs have typically not been used as inputs for 3D models previously because of concerns about image resolution and accuracy. X-ray radiographs are 2D representations of 3D space. As such, a 2D X-ray radiograph necessarily distorts the image subject relative to the actual object that exists in three dimensions. Furthermore, the object through which the X-ray passes can deflect the path of the X-ray as it travels from the X-ray source 21 (typically the emitter or anode of the X-ray machine; see FIG. 11) to the X-ray detector 33 (which may include by non-limiting example, X-ray image intensifiers, phosphorus materials, flat panel detectors “FPD” (including indirect conversion FPDs and direct conversion FPDs), or any number of digital or analog X-ray sensors or X-ray film; see FIG. 11). Defects in the X-ray machine (see 1800, FIG. 11) itself or in its calibration can also undermine the usefulness of X-ray photogrammetry and 3D model reconstruction. Additionally, emitted X-ray photons have different energies. As the X-rays interact with the matter placed between the X-ray source 21 and the detector 33, noise and artifacts can be produced in part because of Compton and Rayleigh scattering, the photoelectric effect, extrinsic variables in the environment or intrinsic variables in the X-ray source 21, X-ray detector 33, and/or processing units 597 or displays 19.

Moreover, in a single 2D image, the 3D data of the actual subject is lost. As such, there is no data that a processor 597 or computer platform 500 generally can use from a single 2D image to reconstruct a 3D model of the actual 3D object. For this reason, CT scans, MRIs, and other imaging technologies that preserve third dimensional data were often preferred inputs for reconstructing models of one or more target orthopedic joints 200 (i.e., reconstructing a 3D model from actual 3D data generally resulted in more accurate, higher resolution models). However, certain exemplary embodiments of the present disclosure that are discussed below overcome these issues by using deep learning networks to improve the accuracy of reconstructed 3D models generated from X-ray input images.

It is contemplated that once the system is calibrated as discussed below, new tissue-penetrating images (i.e., less than the number of input images needed to calibrate the system) can be taken intraoperatively to update the reconstructed model of the operative area (e.g., to refresh the position of the identified component of the endoprosthetic implant related to another component of an endoprosthetic implant or relative to an identified orthopedic element). In other exemplary embodiments, the same number of new tissue-penetrating images as the number of input images chosen to calibrate the system can be used to refresh the position of the component of the endoprosthetic implant relative to another component of an endoprosthetic implant, or relative to and identified orthopedic element in the system.

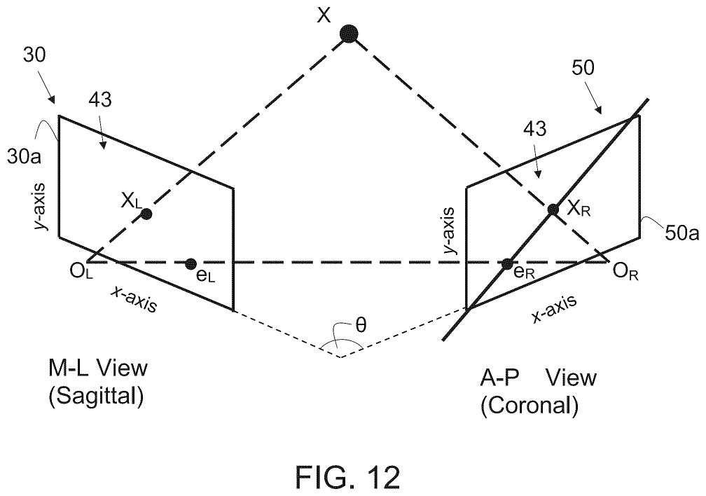

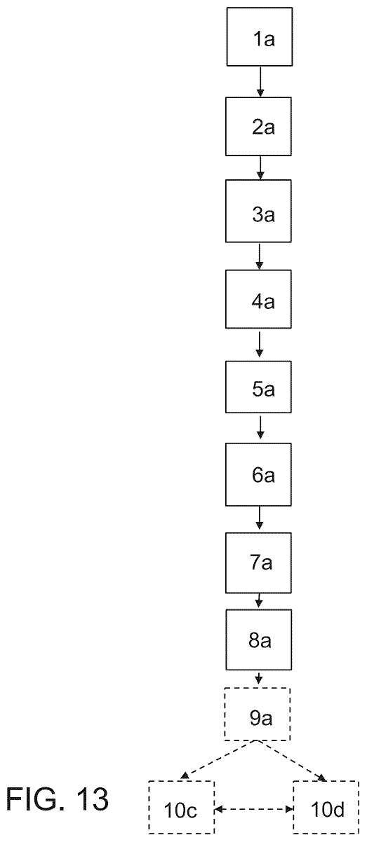

FIG. 13 is a flow chart outlining the steps of an exemplary method for ascertaining a position of an orthopedic element in space, which can then be used to calculate desired alignment angles 235. The method comprises: step 1a calibrating a tissue-penetrating machine, such as a radiographic imaging machine 1800 to determine a mapping relationship between image points (e.g., XL, XR; FIG. 12) and corresponding space coordinates (e.g., x and y coordinates; FIG. 12) to define spatial data 43, step 2a capturing a first image 30 (FIG. 12) of a target orthopedic joint 200 using a radiographic imaging technique, wherein the first image 30 defines a first reference frame 30a, step 3a capturing a second image 50 (FIG. 12) of the target orthopedic joint 200 using the radiographic imaging technique, wherein the second image 50 defines a second reference frame 50a, and wherein the first reference frame 30a is offset from the second reference frame 50a at an offset angle θ, step 4a mapping image points (e.g., XL, XR) from the first input image 30 and the second input image 50 using the mapping relationship of the calibrating step 1a to define spatial data 43, step 5a projecting the spatial data 43 from the first image 30 of the target orthopedic joint 200 and the spatial data 43 from the second image 50 of the target orthopedic joint 200 to define volume data 61 (FIG. 4), step 6a using a deep learning network to identify one or more anatomical landmarks (e.g., edges, anatomical components fitting a learned profile) in the spatial data 43 or the volume data 61 to isolate an anatomical component (e.g., distal femur 105, proximal tibia 110, proximal fibula 111, or soft tissue (e.g., articular cartilage, tendons, ligaments (e.g., the MCL, LCL, ACL, PCL when the target orthopedic joint 200 is a knee joints), muscle, etc.)) from the spatial data 43 or the volume data 61 of the target orthopedic joint 200 to thereby define an identified orthopedic joint component 200a, step 7a applying a mask to the identified orthopedic joint component 200a, wherein the spatial data 43 comprising image points (e.g., XL, XR) disposed within a masked area of either the first image 30 or the second image 50 have a first value and wherein the spatial data 43 comprising image points (e.g., XL, XR) disposed outside of a masked area of either the first image 30 or the second image 50 have a second value, wherein the first value is different from the second value, and step 8a identifying one or more alignment angles 235 from spatial data 43 or volume data 61 from the identified orthopedic joint component 200a or from adjacent identified orthopedic joint components 200a. In other exemplary embodiments, the exemplary method can further comprise step 9a calculating a value an alignment angle 235 and optionally step 10c classifying the identified orthopedic component 200a or the identified orthopedic joint based on the value of the alignment angle 235. In further exemplary embodiments, the exemplary method can further comprise step 10d recommending a type of surgical procedure based on either the value of the alignment angle 235 or the classification of the identified orthopedic component or the classification of the identified orthopedic joint 200a. It will be appreciated that the above steps need not necessarily be performed sequentially. Combinations and permutations with other exemplary methods are considered to be within the scope of this disclosure.

FIG. 11 is a schematic representation of an exemplary system comprising a radiographic imaging machine 1800 comprising an X-ray source 21, such as an X-ray tube, a filter 26, a collimator 27, and a detector 33. In FIG. 11, the radiographic imaging machine 1800 is shown from the top down. The depicted radiographic imaging machine 1800 is a type of tissue-penetrating imaging machine. A patient 1 is disposed between the X-ray source 21 and the detector 33. The radiographic imaging machine 1800 may be mounted on a rotatable gantry 28. The radiographic imaging machine 1800 may take a first radiographic input image 30 of the patient 1 from a first reference frame 30a. The gantry 28 may then rotate the radiographic imaging machine 1800 by an offset angle θ. The radiographic imaging machine 1800 may then take the second radiographic input image 50 from the second reference frame 50a. It will be appreciated that other exemplary embodiments can comprise using multiple input images taken at multiple offset angles θ. For example, in a hip arthroplasty, the radiographic imaging machine 1800 may be further rotated (or the patient rotated) to capture a third radiographic input image from a third reference frame. In such embodiments, the offset angle θ may be less than or greater than 90° between adjacent input images.

It will be appreciated that the offset angle need not be exactly 90 degrees in every embodiment. An offset angle having a value within a range that is plus or minus 45 degrees is contemplated as being sufficient. In other exemplary embodiments, an operator may take more than two images of the orthopedic element using a radiographic imaging technique. It is contemplated that each subsequent image after the second image can define a subsequent image reference frame. For example, a third image can define a third reference frame, a fourth image can define a fourth reference frame, the nth image can define an nth reference frame, etc.

In other exemplary embodiments comprising three input images and three distinct reference frames, each of the three input images may have an offset angle θ of about 60 degrees relative to each other. In some exemplary embodiments comprising four input images and four distinct reference frames, the offset angle θ may be 45 degrees from an adjacent reference frame. In an exemplary embodiment comprising five input images and five distinct reference frames, the offset angle θ may be about 36 degrees from the adjacent reference frame. In exemplary embodiments comprising n images and n distinct reference frames, the offset angle θ can be 180/n degrees.

It is further contemplated that embodiments involving multiple images, especially more than two images, do not necessarily have to have regular and consistent offset angles θ. For example, an exemplary embodiment involving four images and four distinct reference frames may have a first offset angle θ1 at 85 degrees, a second offset angle θ2 at 75 degrees, a third offset angle θ3 at 93 degrees, and a fourth offset angle θ4 at 107 degrees. All offset angles θ from 1 degree to 359 degrees are considered to be within the scope of this disclosure.

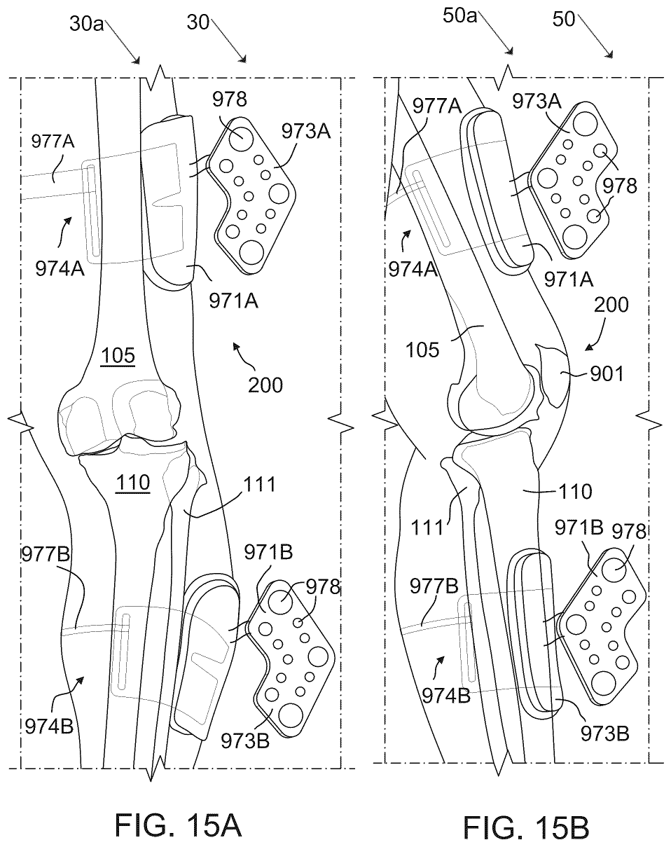

With respect to method step 1a of FIG. 13, FIGS. 15A and 15B illustrate an example of how a tissue-penetrating machine, such as a radiographic imaging machine 1800 can be calibrated to determine a mapping relationship between image points (e.g., XL, XR; FIG. 12) and corresponding space coordinates (e.g., x and y coordinates; FIG. 12) to define spatial data 43 in exemplary photogrammetry embodiments. FIGS. 15A and 15B depict calibration jigs 973A, 973B captured in first and second 2D input images 30, 50 relative to the target orthopedic joint 200. In these figures, the target orthopedic joint 200 is a knee joint comprising multiple orthopedic components. These multiple orthopedic components include the distal aspect of the femur 105 and the proximal aspect of the tibia 110 that comprise a knee joint. The proximal fibula 111 is another orthopedic component imaged in FIGS. 15A and 15B. The patella 901 is another orthopedic component shown in FIG. 15B.

With respect to step 2a of the method of FIG. 13, FIG. 15A is an example anterior-posterior view of the example target orthopedic joint 200 (i.e., FIG. 15A represents a first image 30 taken from a first reference frame 30a (e.g., a first transverse position)). A first calibration jig 973A is attached to a first holding assembly 974A. The first holding assembly 974A may comprise a first padded support 971A engaged to a first strap 977A. The first padded support 971A is attached externally to the patient's thigh via the first strap 977A. The first holding assembly 974A supports the first calibration jig 973A oriented desirably parallel to the first reference frame 30a (i.e., orthogonal to the detector 33). Likewise, a second calibration jig 973B that is attached to a second holding assembly 974B may be provided. The second holding assembly 974B may comprise a second padded support 971B engaged to a second strap 977B. The second padded support 971B is attached externally to the patient's calf via the second strap 977B. The second holding assembly 974B supports the second calibration jig 973B desirably parallel to the first reference frame 30a (i.e., orthogonal to the detector 33). The calibration jigs 973A, 973B arc desirably positioned sufficiently far away from the subject orthopedic elements 100 such that the calibration jigs 973A, 973B do not overlap any desired boney component of the target orthopedic joint 200.

With respect to step 3a of the method of FIG. 13, FIG. 15B is an example medial-lateral view of the example target orthopedic joint 200 (i.e., FIG. 15B represents a second image 50 taken from a second reference frame 50a (e.g., a second transverse position)). In the depicted example, the medial-lateral reference frame 50a is rotated or “offset” 90° from the anterior-posterior first reference frame 30a. The first calibration jig 973A is attached to the first holding assembly 974A. The first holding assembly 974A may comprise a first padded support 971A engaged to a first strap 977A. The first padded support 971A is attached externally to the patient's thigh via the first strap 977A. The first holding assembly 974A supports the first calibration jig 973A desirably parallel to the second reference frame 50a (i.e., orthogonal to the detector 33). Likewise, a second calibration jig 973B that is attached to a second holding assembly 974B may be provided. The second holding assembly 974B may comprise a second padded support 971B engaged to a second strap 977B. The second padded support 971B is attached externally to the patient's calf via the second strap 977B. The second holding assembly 974B supports the second calibration jig 973B desirably parallel to the second reference frame 50a (i.e., orthogonal to the detector 33). The calibration jigs 973A, 973B are desirably positioned sufficiently far away from the subject orthopedic elements 100 such that the calibration jigs 973A, 973B do not overlap the desired boney orthopedic components. Desired boney orthopedic components is this example would be the femur 105 and the tibia 110 because these are the orthopedic components from which the alignment angles 235 will be ascertained.

It will be appreciated that “orthopedic component,” unless further modified, includes any skeletal structure or associated soft tissue, such as tendons, ligaments, cartilage, and muscle. A non-limiting list of example of “orthopedic components” includes any partial or complete bone from a body, including but not limited to a femur, tibia, pelvis, vertebra, humerus, ulna, radius, scapula, skull, fibula, clavicle, mandible, rib, carpal, metacarpal, tarsal, metatarsal, phalange, or any associated tendon, ligament, tissue, cartilage, or muscle.

The patient can desirably be posited in the standing position (i.e., the leg is in extension) because the knee joint is stable in this orientation (see FIG. 11). Preferably, the patient's distance relative to the imaging machine should not be altered during the acquisition of the input images 30, 50. The first and second images 30, 50 need not capture the entire leg, rather the image can focus on the target orthopedic joint 200 that will be the subject of the surgical procedure.

It will be appreciated that depending upon the target orthopedic joint 200 to be imaged or modeled, only a single calibration jig 973 may be used. Likewise, if the target orthopedic joint 200 extends or if multiple target orthopedic elements 200 extend over a particularly large distance (e.g., a spine), more than two calibration jigs may be used.

Each calibration jig 973A, 973B is desirably of a known size. Each calibration jig 973A, 973B desirably has at least four or more calibration points 978 distributed throughout. The calibration points 978 are distributed in a known pattern in which the distance from one point 978 relative to the others is known. The distance from the calibration jig 973 to component of the target orthopedic joint 200 can also be desirably known. For calibration of an X-ray photogrammetry system, the calibration points 978 may desirably be defined by metal structures on the calibration jig 973. Metal typically absorbs most X-ray beams that contact the metal. As such, metal typically appears very brightly relative to material that absorbs less of the X-rays (such as air cavities or adipose tissue). Common example structures that define calibration points include reseau crosses, circles, triangles, pyramids, and spheres.

These calibration points 978 can exist on a 2D surface of the calibration jig 973, or 3D calibration points 978 can be captured as 2D projections from a given image reference frame (e.g., 30a, 50a). In either situation, the 3D coordinate (commonly designated the z coordinate) can be set to equal zero for all calibration points 978 captured in the image. The distance between each calibration point 978 is known. These known distances can be expressed as x, y coordinates on the image sensor/detector 33. To map a point in 3D space to a 2D coordinate pixel on a sensor 33, the dot product of the detector's calibration matrix, the extrinsic matrix, and the homologous coordinate vector of the real 3D point can be used. This permits the real world coordinates of a point in 3D space to be mapped relative to calibration jig 973. Stated differently, this generally permits the x, y coordinates of the real point in 3D space to be transformed accurately to the 2D coordinate plane of the image detector's sensor 33 to define spatial data 43 (see FIG. 4).

The above calibration method is provided as an example. It will be appreciated that all methods suitable for calibrating an X-ray photogrammetry system are considered to be within the scope of this disclosure. A non-limiting list of other X-ray photogrammetry system calibration methods include the use of a reseau plate, the Zhang method, the bundle adjustment method, direct linear transformation methods, maximum likelihood estimation, a k-nearest neighbor regression approach (“kNN”), other deep learning methods, or combinations thereof.

Although at least two input images 30, 50 are technically required for calibrating the exemplary systems described herein, at least three input images can be desirable when the input images are radiographic input images and wherein the target operative area involves a contralateral joint that cannot be easily isolated from radiographic imaging. For example, the pelvis comprises contralateral acctabula. A direct medial-lateral radiograph of the pelvis would show both the acctabulum that is proximal to the detector 33 and the acetabulum that is distal to the detector 33. However, because of the positioning of the pelvis relative to the detector 33 and because a single 2D radiograph lacks 3D data, the relative acetabula will appear superimposed upon one another and it would be difficult for a person, processor 597, or other computational machine 500 to distinguish which is the proximal and which is the distal acctabulum.

To address this issue, at least three input images can be used. In one exemplary embodiment, the first input image 30 can be a radiograph that captures an anterior-posterior perspective of the operative area (i.e., an example of a first reference frame 30a). For the second input image 50, the patient or the detector 33 can be rotated clockwise (which can be designated by a positive degree) or counterclockwise (which can be designated by a negative degree) relative to the patient's orientation for the first input image 30. For example, for the second input image 50, the patient may be rotated plus or minus 45° from the patient's orientation in the first input image 30. Likewise, the patient may be rotated clockwise or counterclockwise relative to the patient's orientation for the first input image 30. For example, for the third input image (not depicted), the patient may be rotated plus or minus 45° relative to the patient's orientation in the first input image 30. It will be appreciated that if the second input image 50 has a positive offset angle (e.g., +45°) relative to the orientation of the first input image 30, the third input angle desirably has a negative offset angle (e.g., −45°) relative to the orientation of the first input image 30 and vice versa.

In exemplary embodiments, the principles or epipolar geometry can be applied to at least three input images taken from at least three different reference frames to calibrate exemplary systems or to perform the calibration step of exemplary methods.

FIG. 12 illustrates basic principles of epipolar geometry than can be used to convert spatial data 43 from the respective input images 30, 50 into volume data 61. It will be appreciated that the spatial data 43 is defined by a collection of image points (e.g., XL, XR) mapped to corresponding space coordinates (e.g., x and y coordinates) for a given input image 30, 50 (see step 4a of the method of FIG. 13).

FIG. 12 is a simplified schematic representation of a perspective projection described by the pinhole camera model. FIG. 12 conveys basic concepts related to computer stereo vison, but it is by no means the only method by which 3D models can be reconstructed from 2D stereo images. In this simplified model, rays emanate from the optical center (i.e., the point within a lens at which the rays of electromagnetic radiation (e.g., visible light, X-rays, etc.) from the subject object are assumed to cross within the imaging machine's sensor or detector array 33 (FIG. 11). The optical centers are represented by points O_{L}, O_{R} in FIG. 12. In reality, the image plane (see 30a, 50a) is usually behind the optical center (e.g., O_{L}, O_{R}) and the actual optical center is projected onto the detector array 33 as a point, but virtual image planes (see 30a, 50a) are presented here for illustrating the principles more simply.

The first input image 30 is taken from a first reference frame 30a, while the second input image 50 is taken from a second reference frame 50a that is different from the first reference frame 30a. Each image comprises a matrix of pixel values. The first and second reference frames 30a, 50a are desirably offset from one another by an offset angle θ. The offset angle θ can represent the angle between the x-axis of the first reference frame 30a relative to the x-axis of the second reference frame 50a. Stated differently, the angle between the orientation of the target orthopedic joint 200 in the first image 30 and the target orthopedic joint 200 in the second image 50 can be known as the “offset angle.”

Point e_{L} is the location of the second input image's optical center O_{R} on the first input image 30. Point e_{R} is the location of the first input image's optical center O_{L}, on the second input image 50. Points e_{L} and e_{R} are known as “epipoles” or epipolar points and lie on line O_{L}−O_{R}. The points X, O_{L}, O_{R} define an epipolar plane.

Because the actual optical center is the assumed point at which incoming rays of electromagnetic radiation from the subject object cross within the detector lens, in this model, the rays of electromagnetic radiation can be imagined to emanate from the optical centers O_{L}, O_{R} for the purpose of visualizing how the position of a 3D point X in 3D space can be ascertained from two or more input images 30, 50 captured from a detector 33 of known relative position. If each point (e.g., X_{L}) of the first input image 30 corresponds to a line in 3D space, then if a corresponding point (e.g., X_{R}) can be found in the second input image, then these corresponding points (e.g., X_{L}, X_{R}) must be the projection of a common 3D point X. Therefore, the lines generated by the corresponding image points (e.g., O_{L}−X_{L}, O_{R}−X_{R}) must intersect at 3D point X. In general, if the value of X is calculated for all corresponding image points (e.g., X_{L}, X_{R}) in the two or more input images 30, 50, a 3D volume comprising volume data 61 can be reproduced from the two or more input images 30, 50. The value of any given 3D point X can be triangulated in a variety of ways. A non-limiting list of example calculation methods include the mid-point method, the direct linear transformation method, the essential matrix method, the line-line intersection method, and the bundle adjustment method. Furthermore, in certain exemplary embodiments, a deep learning network can be trained on a set of input images to establish a model for determining the position of a given point in 3D space based upon two or more input images of the same subject, wherein the first input image 30 is offset from the second input image 50 at an offset angle θ. It will further be appreciated that combinations of any of the above methods are within the scope of this disclosure.

It will be appreciated that “image points” (e.g., X_{L}, X_{R}) described herein may refer to a point in space, a pixel, a portion of a pixel, or a collection of adjacent pixels. It will also be appreciated that 3D point X as used herein can represent a point in 3D space. In certain exemplary applications, 3D point X may be expressed as a voxel, a portion of a voxel, or a collection of adjacent voxels.

FIG. 4 further depicts an example of how a deep learning network can be modeled and used to identify at least two bones (e.g., orthopedic components) that comprise the target orthopedic joint 200 to define an identified orthopedic joint 200a. The top left block in the model of FIG. 4 is an example illustration of projecting spatial data 43 from the first image 30 of the target orthopedic joint 200 and the spatial data 43 from the second image of the target orthopedic joint 200 to define volume data 61 (step 5a of the method of FIG. 13, see also FIG. 12).

FIG. 4 further illustrates an example of how this deep learning model can be adapted to identify areas of bone or soft tissue loss on the identified orthopedic joint 200a to define an identified loss area 79a. The same model, or in other embodiments, another model can be used to identify alignment angles 235a of the identified orthopedic joint 200a. An adjustment algorithm can then be applied to the identified loss area 79a to replace the identified loss area 79a with a reconstructed area 79b to thereby define a reconstructed orthopedic joint 200b (see FIG. 3B and FIG. 11). The model, or in other embodiments, another model, can then be used to adjust the values of the identified alignment angles 235a to reflect the values of the identified alignment angles 235a on the repositioned reconstructed orthopedic joint 200b.

In certain exemplary embodiments, the adjustment algorithm for reconstructing an identified area of bone or soft tissue loss 79a can be a curve fitting algorithm. An exemplary curve fitting algorithm may involve interpolation or smoothing. In other exemplary embodiments, the curve fitting algorithm may be used to extrapolate the position of the pre-diseased articular surface of the bone or soft tissue. In other exemplary embodiments, the adjustment algorithm can identify the dimensions of a non-worn contralateral orthopedic element 100, such as a non-worn contralateral condyle. The adjustment algorithm can add the surface of the non-worn orthopedic element to the corresponding area of bone loss on the worn orthopedic element 100 to calculate and replace the volume of the identified loss area 79a.

It will be appreciated that the model described with reference to FIG. 4 can be used with either photogrammetry embodiments or embodiments that start with three dimensional data sets, although the below detailed examples will describe the model's use with a photogrammetry embodiment. In the depicted example, the input data set 10 is desirably an anterior to posterior image of the target orthopedic joint 200. When the knee is the target orthopedic joint 200, it is contemplated that the exemplary systems and methods described herein may obviate the need for a long leg image of the target leg and knee.

In exemplary systems and methods for a processor 597 identifying an orthopedic joint 200a and/or a component of an orthopedic joint or of an endoprosthetic implant and in exemplary systems and methods for ascertaining a position of an orthopedic joint, a component of an orthopedic joint, an endoprosthetic implant, or a component thereof in space using a deep learning network, wherein the deep learning network is a CNN, a detailed example of how the CNN can be structured and trained is provided. All architecture of CNNs are considered to be within the scope of this disclosure. Common CNN architectures include, by way of example, LeNet, GoogLeNet, AlexNet, ZFNet, ResNet, and VGGNet.

Preferably, the methods disclosed herein may be implemented on a computer platform (see 500) having hardware such as one or more processors 597, such as central processing units (CPU) or graphic processing units (GPU), a random access memory (RAM), and input/output (I/O) interface(s).

FIG. 4 is a schematic representation of a CNN that further illustrates how the CNN can be used to identify the edges of a target orthopedic joint 200 to define an identified orthopedic joint 200a (see step 6a of the exemplary method of FIG. 13). Without being bound by theory, it is contemplated that a CNN may be desirable for reducing the file size of the input data set 10 without losing features that are necessary to identify the desired orthopedic joint 200 or its surface topography. In the depicted example, the input data set 10 comprises at least two radiographic images of the target orthopedic joint 200 taken at a known offset angle. Using principles of epipolar geometry, and the calibration step described above, the two or more offset 2D input images 30, 50 are projected along the acute offset angle and a 3D volume can be created from the input images 30, 50. A filter (also known as a kernel 69) is shown disposed in the input data set 10. The kernel 69 is a tensor (i.e., a multi-dimensional array) that defines a filter or function (this filter or function is sometimes known as the “weight” given to the kernel). In the depicted embodiment, the kernel tensor 69 is three dimensional; however, it will be appreciated that in other exemplary embodiments, the kernel tensor 69 can be two-dimensional. The filter or function that comprises the kernel 69 can be programed manually or learned through the CNN, RNN, or other deep learning network. In the depicted embodiment, the kernel 69 is a 3×3×3 tensor although all tensor sizes and dimensions are considered to be within the scope of this disclosure, provided that the kernel tensor size is less than the size of the input tensor (i.e., the input data set 10).

Each cell or pixel of the kernel 69 has a numerical value. These values define the filter or function of the kernel 69. A convolution or cross-correlation operation is performed between the two tensors. In FIG. 4, the convolution is represented by the path 76. The path 76 that the kernel 69 follows is a visualization of the mathematical convolution operation. Following this path 76, the kernel 69 eventually and sequentially traverses the entire space of the input tensor (e.g., the input image data set 10). The goal of this operation is to extract features from the input tensor.

Convolution layers 72 typically comprise one or more of the following operations: a convolution stage 67, a detector stage 68, and a pooling stage 58. Although these respective operations are represented visually in the first convolution layer 72a in FIG. 4, it will be appreciated that the subsequent convolution layers 72b, 72c, etc. may also comprise one or more or all of the convolution stage 67, detector stage 68, and pooling layer 58 operations or combinations or permutations thereof. Furthermore, although FIG. 4, depicts five convolution layers 72a, 72b, 72c, 72d, 72e of various resolutions, it will be appreciated that more or less convolution layers may be used in other exemplary embodiments.

In the convolution stage 67, the kernel 69 is sequentially multiplied by multiple patches of pixels or voxels in the input data set 10. The patch of pixels extracted from the data is known as the receptive field. The multiplication of the kernel 69 and the receptive field comprises an element-wise multiplication between each pixel of the receptive field and the kernel 69. After multiplication, the results are summed to form one element of a convolution output. This kernel 69 then shifts to the adjacent receptive field and the element-wise multiplication operation and summation continue until all the pixels of the input tensor have been subjected to the operation.

Until this stage, the input image data set 10 of the input tensor has been linear. To introduce non-linearity to this data, a nonlinear activation function is then employed. Use of such a non-linear function marks the beginning of the detector stage 68. A common non-linear activation function is the Rectified Linear Unit function (“ReLU”), which is given by the function:

$$Re LU ( x ) = \{ \begin{matrix}0 , & if x < 0 \\ x , & if x \geq 0\end{matrix} \}$$

When used with bias, the non-linear activation function serves as a threshold for detecting the presence of the feature extracted by the kernel 69. For example, applying a convolution or a cross-correlation operation between the input tensor and the kernel 69, wherein the kernel 69 comprises a low level edge filter in the convolution stage 67 produces a convolution output tensor. Then, applying a non-linear activation function with a bias to the convolution output tensor will return a feature map output tensor. The bias is sequentially added to each cell of the convolution output tensor. For a given cell, if the sum is greater than or equal to 0 (assuming ReLU is used in this example), then the sum will be returned in the corresponding cell of the feature map output tensor. Likewise, if the sum is less than 0 for a given cell, then the corresponding cell of the feature map output tensor will be set to 0. Therefore, applying non-linear activation functions to the convolution output behaves like a threshold for determining whether and how closely the convolution output matches the given filter of the kernel 69. In this manner, the non-linear activation function detects the presence of the desired features from the input image data set 10 (e.g., an edge, a pattern of edges that the network has been trained to recognize, which can include, but is not limited to edges that form a recognized anatomical feature of the target orthopedic joint 200). It will be appreciated that anatomical features of the target orthopedic joint 200 will vary based on what the target orthopedic joint 200 is. In embodiments in which the target orthopedic joint 200 is a knee, examples of anatomical features include the adductor tubercle, medial or lateral femoral condyles or epicondyles, popliteal groove, intercondylar fossa, patella, patellar apex, tibial tuberosity, the lateral tibial (Gerdy) tubercle, medial or lateral tibial hemicondyles or tubercles, intercondylar eminence, fibula head, resected portions of any of the foregoing, ACL, PCL, MCL, LCL, or patellar tendon.

All non-linear activation functions are considered to be within the scope of this disclosure. Other examples include the Sigmoid, Tan H, Leaky ReLU, parametric ReLU, Softmax, and Switch activation functions.

However, a shortcoming of this approach is that the feature map output of this first convolutional layer 72a records the precise position of the desired feature (in the above example, an edge or pattern of edges). As such, small movements of the feature in the input data set 10 will result in a different feature map. To address this problem and to reduce computational power, down sampling can be used to lower the resolution of the input data set 10 while still preserving the significant structural elements. This can be especially useful when an exemplary system is being trained with multiple data sets or when the input data set 10 comprises sequential tissue-penetrating images, such as in video taken intraoperatively from a C-arm radiographic imaging machine. Down sampling can be achieved by changing the stride of the convolution along the input tensor. Down sampling is also achieved by using a pooling layer 58.

Valid padding may be applied to reduce the dimensions of the convolved tensor (see 72b) compared to the input tensor (see 72a). A pooling layer 58 is desirably applied to reduce the spatial size of the convolved data, which decreases the computational power required to process the data. Common pooling techniques, including max pooling and average pooling may be used. Max pooling returns the maximum value of the portion of the input tensor covered by the kernel 69, whereas average pooling returns the average of all the values of the portion of the input tensor covered by the kernel 69. Max pooling can be used to reduce image noise.

In certain exemplary embodiments, a fully connected layer can be added after the final convolution layer 72e to learn the non-linear combinations of the high level features (such as for example, the profile of an imaged distal femur 105, the profile of a proximal tibia 110, or the collective edges of the orthopedic joint 200, or the profile of a target anatomical feature) represented by the output of the convolutional layers. In this manner, when used on an orthopedic joint 200, the above description of a CNN type deep learning network is one example of how a deep learning network can be “configured to identify” an orthopedic joint 200 to define an “identified orthopedic joint” 200a.

The top half of FIG. 4 represents compression of the input image data set 10, whereas the bottom half represents decompression until the original size of the input image data set 10 is reached. The output feature map of each convolution layer 72a, 72b, 72c, etc. is used as the input for the following convolution layer 72b, 72c, etc. to enable progressively more complex feature extraction. For example, the first kernel 69 may detect edges, a kernel in the first convolution layer 72b may detect a collection of edges in a desired orientation, a kernel in a third convolution layer 72c may detect a longer collection of edges in a desired orientation, etc. This process may continue until the entire profile of the target orthopedic joint 200 is detected and identified by a downstream convolution layer 72. By way of another example, subsequent convolutions layers may be trained to identify an anatomical feature of the target orthopedic joint.

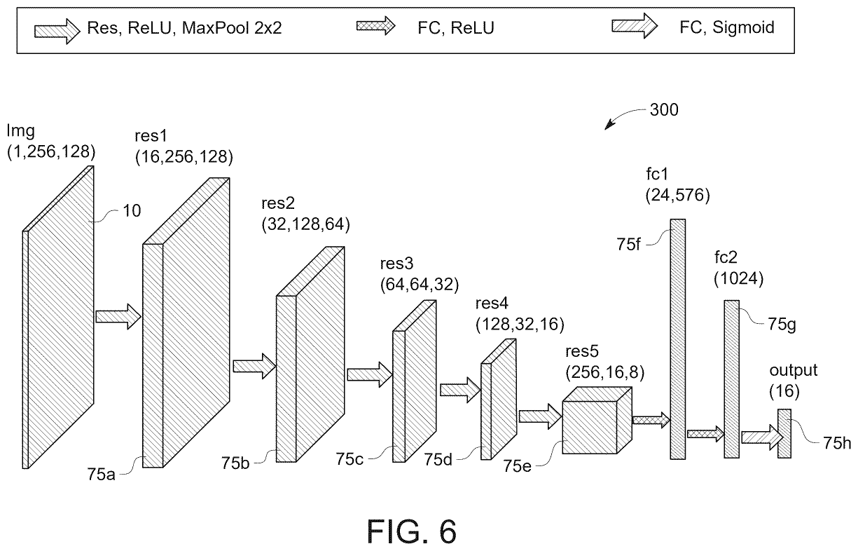

The bottom half of FIG. 4 up-samples (i.e., expands the spatial support of the lower resolution feature maps. A de-convolution operation is performed in order to increase the size of the input for the next downstream convolutional layer (see 72c, 72d, 72e). For the final convolution layer 72e, a convolution can be employed with a 1×1×1 kernel 69 to produce a multi-channel output volume 59 that is the same size as the input volume 61. Each channel of the multi-channel output volume 59 can represent a desired extracted high level feature. This can be followed by a Softmax activation function to detect and identify the target orthopedic joint 200. The alignment angles 235 may then be identified. FIGS. 6-8 illustrate exemplary deep learning models that can be configured to identify the desired alignment angles 235. Combining these models desirably produces several output channels. For example, the depicted embodiment may comprise seven output channels numbered 0, 1, 2, 3, 4, 5, 6 wherein channel 0 represents identified background volume, channel 1 represents the identified distal femur 105, channel 2 represents the identified proximal tibia 110, channel 3 represents the identified proximal fibula 111, channel 4 represents the identified LDFA (i.e., a type of alignment angle 235), channel 5 represents the identified MPTA (i.e., a type of alignment angle 235), and channel 6 represents the identified mHKA angle (i.e., a type of alignment angle 235) (see steps 8a, 9a, 10a, and 10b of the exemplary method of FIG. 13).

In other exemplary embodiments, additional channels may be used to represent the identified loss area 79a, the reconstructed area 79b, the reconstructed orthopedic joint 200b, which in the depicted example would comprise at least a reconstructed distal femur and desirably a reconstructed proximal tibia, and reconstructed alignment angles 235b based on the reconstructed orthopedic joint 200b. It will be appreciated that a reconstructed pre-diseased constitutional joint line can be a type of reconstructed alignment angle that is ascertained from the reconstructed orthopedic joint 200b. Therefore, in one such exemplary manner, an orthopedic image processing system can be said to be “configured to return a predicted constitutional joint line based on the reconstructed alignment angle.”

It will be appreciated that less output channels or more output channels may be used in other exemplary embodiments. It will also be appreciated that the provided output channels may represent different target orthopedic joints 200, components of endoprosthetic implants, trial components of endoprosthetic implants, alignment angles 235, markers, or surgical instruments than those listed here.

The above described embodiment is one example of how a processor 597 that utilizes the above describe CNN can be said to perform operations, the operations comprising: identifying at least two bones comprising a target orthopedic 200 joint to define an identified orthopedic joint 200a; identifying an area of bone or soft tissue loss 79 (FIG. 3A) in the identified orthopedic joint 200a to define an identified loss area 79a, applying an adjustment algorithm to replace the identified loss area with a reconstructed area to thereby define a reconstructed orthopedic joint 200b (FIG. 3B, see also the system of FIG. 11), and identifying an alignment angle 235 of the reconstructed orthopedic joint 200b to define a reconstructed alignment angle 235b.

When used on a component of an endoprosthetic implant or subcomponents thereof, the above description of a CNN type deep learning network is one example of how a deep learning network can be “configured to identify” a component of an endoprosthetic implant (or subcomponents thereof) to define an identified component of the endoprosthetic implant. When used on an endoprosthetic implant, the above description of a CNN type deep learning network is one example of how a deep learning network can be “configured to identify” an endoprosthetic implant to define an “identified endoprosthetic implant.” It will be further understood that when applied to multiple orthopedic joints, multiple components of endoprosthetic implants, multiple endoprosthetic implants, or combinations thereof, the above description of a CNN type deep learning network is one example of how a deep learning network can be “configured to identify” multiple orthopedic elements, multiple components of endoprosthetic implants, subcomponents thereof, multiple endoprosthetic implants, or combinations thereof as the case may be. The same applies mutandis mutatis to systems or deep learning networks that are “configured to identify” any trial components of endoprosthetic implants, alignment angles 235, orientation information, predicted constitutional joint lines based on the position of anatomical markers of a reconstructed joint in any manner that is within the scope of this disclosure, markers, surgical instruments, or combinations thereof. Other deep learning network architectures known or readily ascertainable by those having ordinary skill in the art are also considered to be within the scope of this disclosure.

In embodiments wherein any of the first input image, the second input image, or additional input images are radiographic X-ray images (including, but not limited to fluoroscopic radiographic images), training a CNN can present several challenges. By way of comparison, CT scans typically produce a series of images of the desired volume. Each CT image that comprises a typical CT scan can be imagined as a segment of the imaged volume. From these segments, a 3D model can be created relatively easily by adding the area of the desired element as the element is depicted in each successive CT image. The modeled element can then be compared with the data in the CT scan to ensure accuracy. One drawback of CT scans is that CT scans expose the patient to excessive amounts of radiation (about seventy times the amount radiation of one traditional radiograph).

By contrast, radiographic imaging systems typically do not generate sequential images that capture different segments of the imaged volume; rather, all of the information of the image is flattened on the 2D plane. Additionally, because a single radiographic image 30 inherently lacks 3D data, it is difficult to check the model generated by the epipolar geometry reconstruction technique described above with the actual geometry of the target orthopedic joint 200. To address this issue, the CNN can be trained with CT images, such as digitally reconstructed radiograph (“DRRs”) images. By training the deep learning network in this way, the deep learning network can develop its own weights (e.g., filters) for the kernels 69 to identify a desired orthopedic joint 200 or surface topography of a target orthopedic joint 200. Because X-ray radiographs have a different appearance than DRRs, image-to-image translation can be performed to render the input X-ray images to have a DRR-style appearance. An example image-to-image translation method is the Cycle-GAN image translation technique. In embodiments in which image-to-image style transfer methods are used, the style transfer method is desirably used prior to inputting the data into a deep learning network for feature detection.

The above examples are provided for illustrative purposes and are in no way intended to limit the scope of this disclosure. All methods for generating a 3D model of the target orthopedic joint 200 from 2D radiographic images of the same target orthopedic joint 200 taken from at least two transverse positions (e.g., 30a, 50a) are considered to be within the scope of this disclosure.

In exemplary embodiments, the dimensions of the identified orthopedic joint 200a or of the components of an endoprosthetic implant assembly can be mapped to spatial data 43 that is derived from the input data set 10 to ascertain the position of the identified alignment angles 235a or the component of the endoprosthetic implant assembly relative to the identified orthopedic joint 200a. If this information is displayed to the surgeon and is updated in real time or near real time based upon the surgeon's repositioning of the implant component relative to the identified orthopedic element, the surgeon can use exemplary embodiments in accordance with this disclosure to accurately align the implant component relative to the identified orthopedic element.

It will be appreciated that in certain exemplary embodiments, the deep learning network can be the same deep learning network that has been separately trained to perform the discrete tasks (e.g., identification of the orthopedic joint 200 to define an identified orthopedic joint 200a, applying a mask to the identified orthopedic joint 100a, identifying the one or more alignment angles 235 on the identified orthopedic joint 200a using boney landmarks, etc.). In other exemplary embodiments, a different deep learning network can be used to perform one or more of the discrete tasks.

It is contemplated that by having a 3D model of the identified orthopedic joint 200a, the orientation of a seated endoprosthetic implant may be visualized before the endoprosthetic implant is implanted. In this manner, the display 19 may return a ‘functional alignment score’ in which the position of the implant is displayed as a “best fit” percentage in which a percentage reaching or close to 100% reflects the alignment of an identified component of an endoprosthetic implant (e.g., a femoral component) relative to the reference distal femur 105 of the identified orthopedic joint 200a that is displayed and oriented at a position that the exemplary system calculates to be the best fit for a functionally aligned implant component. In this manner, an exemplary system described herein may realize the benefits of both an acceptably approximated reconstructed pre-diseased joint line and the orientation of the implant in a mechanically stable position.

Referring back to FIG. 11, after the input images 30, 50 have been captured, a transmitter 29 then transmits the first input image 30 and the second input image 50 to computational machine 500 (see also FIG. 5). The computational machine can comprise a processor 597, and memory storing instructions 582 that, when executed by the processor 597, cause the processor 597 to perform operations. These operations can include using a deep learning network to identify the target orthopedic joint 200 or orthopedic components thereof, which may include a bone, a soft tissue, a component of an endoprosthetic implant, subcomponent of a component of an endoprosthetic implant, or the endoprosthetic implant itself, an instrument, a marker, or combinations or sub-combinations of the foregoing in the manner described above or any manner that is consistent with this disclosure.

FIG. 11 also depicts the output data from the computational machine 500 being transmitted to a display 19. A display 19 can depict the identified orthopedic joint 200a, reconstructed orthopedic joint 200b or subcomponents thereof. In exemplary embodiments, it is contemplated that one or more alignment angles 235 can be superimposed on the identified orthopedic joint 200a, reconstructed orthopedic joint 200b. The superimposition can be calculated and displayed using the mapped spatial data 43 of the identified orthopedic joint 200b or components thereof (e.g., the component of the endoprosthetic implant and/or the identified orthopedic joint onto or into which the component of the endoprosthetic implant can be implanted).

In this manner, the surgeon and others in the operating room can have a near real time visualization of the identified target orthopedic joint 200a or the reconstructed orthopedic joint 200b.

Furthermore, because the spatial data 43 of an identified orthopedic joint 200a and because the spatial data 43 of the reconstructed orthopedic joint 200b can be obtained from exemplary systems described herein, the value of the identified alignment angles 235a can be calculated and further displayed on a display 19 in exemplary system embodiments. For example, a calculated FDFA, MPTA, and mHKA of the reconstructed orthopedic joint 200b (in this case, a patient's knee) can displayed on a display 19. By way of still yet another example, the display 19 may optionally display a “best fit” percentage in which a percentage reaching or close to 100% reflects the alignment of an identified component of an endoprosthetic implant (e.g., a femoral component) relative to a reference orthopedic element (e.g., the distal femur 105 onto which the femoral component is to be implanted).

Although the target joint depicted in FIGS. 3A, 3B, FIG. 11, and FIG. 14 is a knee joint, depicted in extension and as being imaged or displayed in an anterior to posterior direction as if the image were taken along a transverse plane, it will be appreciated that the knee joint could be imaged in flexion, at regular intervals from flexion to extension, or at regular intervals from extension to flexion in any manner described herein. Additionally, it will be appreciated that the target joint can be imaged from any direction provided that the captured data is sufficient in quantity and quality for the captured data to be interpretable by the processor in many manner described herein. Examples of other anatomical planes from which a target joint can be imaged include the sagittal plane, the coronal plane, and the transverse plane.

It will be appreciated that all methods for identifying a target orthopedic joint 200 based on an input data set 10 derived from at least one tissue-penetrating image file are considered to be within the scope of this disclosure. Other exemplary methods to identify the target orthopedic element 200 and the alignment angles 235 are provided with reference to FIGS. 6-10 and 16.

### Non-Photogrammetry Embodiments Involving 2D Input Images

FIG. 6 is a schematic representation of an exemplary residual neural network, wherein the tensors 75a, 75b, 75c, 75d, and 75e comprise a residual connection (res1, res2, res3, etc.) that is configured to identify two or more bones of a target orthopedic joint 200 to define an identified orthopedic joint 200a and/or a reconstructed orthopedic joint 200b and/or an identified or reconstructed alignment angle 235a, 235b of the identified orthopedic joint 200a or of the reconstructed orthopedic joint 200b. Furthermore, it will be appreciated that the exemplary convolutional neural networks described herein, including, but not limited to the aspects configured to identify the target orthopedic joint 200, the reconstructed orthopedic joint 200b, the area of bone or tissue loss 79 and/or the alignment angles 235 may be applied to the identified target orthopedic joint 200a and/or the reconstructed orthopedic joint 200b in isolation or in combination with any of the other methods disclosed herein to identify the target orthopedic joint 200, the reconstructed orthopedic joint 200b, the area of bone or some tissue loss 79 and/or the identified or reconstructed alignment angles 235a, 235b. Any processor 597 that is programed or trained to run an operation that utilizes any described model herein can be said to be “configured to identify an alignment angle 235 of the identified orthopedic joint 200a or of the reconstructed orthopedic joint 200b.” Likewise, any processor 597 that is programed or trained to run an operation that utilizes any described model herein can be said to be “configured to identify” the target orthopedic joint 200, the reconstructed orthopedic joint 200b, an endoprosthetic implant, a surgical instrument, an alignment angle 235, or a component of any of the foregoing as applicable.

For the purposes of illustration and example, the remainder of the detailed description of FIG. 6 will focus on how this exemplary model can be used to identify alignment angles 235 on an identified target orthopedic joint 200a. In the depicted exemplary embodiment, input data set 10 for the model can be a single a 2D tissue-penetrating image. In the depicted example, this input image represents the input data set 10 and has dimensions of 1 pixel by 256 pixels by 128 pixels. For each tensor 75a, 75b, 75c, 75d, and 75e, a residual connection (Res), activation function such as ReLU, MaxPool function, and 2×2 padding can be used to process the upstream tensor. The residual connection used in this exemplary embodiment are given by:

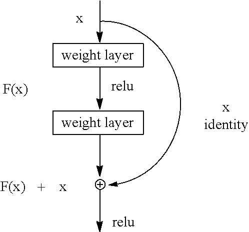

Where x is the input to the residual connection and F(x) represents a “residual function” F(x)=H(x)−x, where H(x) is the underlying function performed by the present subnetwork. Stated differently, F(x)=[the output of the present subnetwork]−[the input of the present subnetwork]. This equation can be rearranged to be written as H(x)=F(x)+x. That is, the residual function can be thought to calculate the difference between the output and the input of a given subnetwork, which can then be used to map the input of the subnetwork to its output. By contrast, the layers of a traditional network are trying to learn the function H(x). Without being bound by theory, it is thought that the use of residual blocks can stabilize training and convergence while reducing overall computing power.

After several residual and pooling operations, the feature maps become smaller in the x and y dimensions, but become larger in the z dimension (compare 75a to 75e). Fully connected (“FC”) layers and the ReLU activation function can be used to up sample tensor 75e into tensors 75f and 75g. An FC layer and a Sigmoid activation function can then be used to produce the output 75h. The output of the example model is 16 values that can describe the lines that comprise the alignment angles 235, which are the coordinates of one point on the line in the form of (x,y), and a vector in the form of (x,y) that represents the direction of the line.

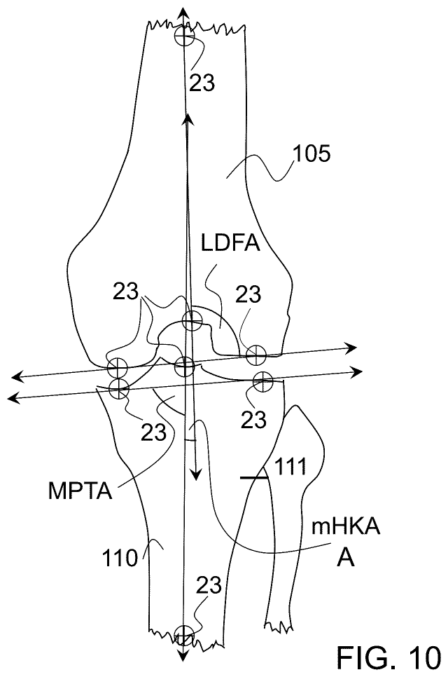

The final tensor 75h comprises 16 values, which denote the four lines that can be used to identify and calculate the alignment angles 235. In exemplary embodiments wherein the target orthopedic joint 200 is a knee joint, a first line can be the distal femur joint line 52 (FIG. 3A), a second line can be the femoral mechanical axis 62 (FIG. 3A), a third line can be the proximal tibial joint line 54 (FIG. 3A), and a fourth line can be the tibial mechanical axis 63 (FIG. 3A). In the model of FIG. 6, each line (e.g., the distal femur joint line 52, the femoral mechanical axis 62, the proximal tibial joint line 54, and the tibial mechanical axis 63 uses four values (see the target points 23 in FIG. 10). The first and second values for each line represent a point on the target anatomical structure. The third and fourth values for each line represent direction. However, for the coordinates of any point on the line, these can take any value in the line, which can make this particular model more difficult to converge.

That is, and without being bound by theory, it is contemplated that the coordinates of a line can be difficult to converge if the underlying anatomical feature of the target orthopedic joint 200 are not identified with a high level of accuracy and precision. This is because the coordinates identified with this model of FIG. 6 can be any point on the identified line and it is contemplated that identified lines would extend beyond the image of the underlying anatomy from which they are first ascertained. This can make checking the accuracy of the identified line, particularly if an identified point in the line does not correspond to a defining anatomical feature of the underlying bone, difficult and error prone.

A solution can be to use this model together with a model that identifies the orthopedic joint or anatomical landmarks thereof (such as any of the models that are within the scope of the present disclosure) to check that the points identified through the model of FIG. 6, which define a given line also correspond to points where the line crosses a designated or identified plane, surface, or anatomical feature of the bone. Once the solution is calculated, the positions of each of the four lines becomes known. Once the positions of each of the four lines become known, the value of each alignment angle 235 can be calculated.

The LDFA is a type of alignment angle 235 that can be defined as the lateral angle formed at the intersection of the distal femur joint line 52 and the femoral mechanical axis 62. The MPTA is a type of alignment angle 235 that can be defined as the medial angle formed at the intersection of the proximal tibia joint line 54 and the tibial mechanical axis 63. The mHKA angle is a type of alignment angle 235 that can be defined as the acute angle formed by the intersection of the femoral mechanical axis 62 and the tibial mechanical axis 63. It will be appreciated that the foregoing are common alignment angles 235 for the knee joint, but that all alignment angles 235 of any target orthopedic joint 200 ascertained through any of the exemplary methods or systems described herein are considered to be within the scope of this disclosure.

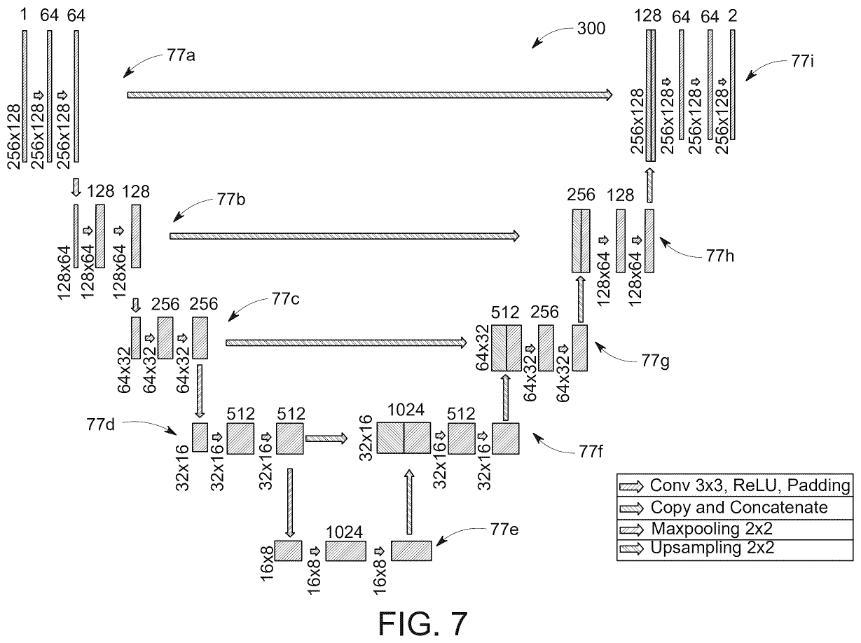

FIG. 7 is a schematic representation of an exemplary U-net convolutional neural network that is configured to identify the target orthopedic joint 200 and that can optionally be further useful to identify an alignment angle 235 of the identified or reconstructed orthopedic joint 200a, 200b to define an identified alignment angle 235a or a reconstructed alignment angle 235b respectively. It is contemplated that this exemplary model can be trained on a first target orthopedic component (e.g., a first bone, such as the distal femur) and can again be separately trained on a second target orthopedic component (e.g., a second bone, such as a proximal tibia). The model is designed to return an output having the same dimensions as the original input data set 10. Collectively, when the model of FIG. 7 is applied to two target orthopedic components, said model can be said to return a total of two outputs-one for each identified orthopedic component. Any processor 597 that is programed to run an operation that utilizes any described model herein, especially with regards to FIG. 4, 6, 7, or 9 can be said to be “configured to identify at least two bones comprising a target orthopedic joint 200 to define an identified orthopedic joint 200a.”

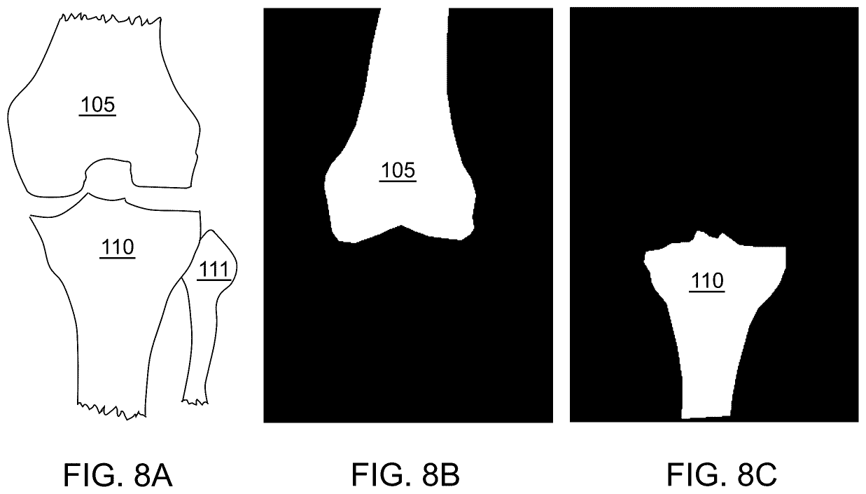

The input data set 10 to this model is 2D tissue-penetrating image (see FIG. 8A). This input data set 10 is processed by several convolutional and pooling operations that are more fully depicted in FIG. 7 itself (and with general reference made to the detailed description of FIG. 4 above), which are the encoding part of the model (i.e., the descending side of U-net CNN).

In particular, the starting 2D tissue-penetrating image is 256 pixels by 128 pixels. A series of convolutions with a 3×3 filter tensor, ReLU activation function, and padding is used in each of the convolution layers 77a, 77b, 77c, 77d, 77e, 77f, 77g, 77h, 77i. In the encoding part of the model, i.e., convolution layers 77a, 77b, 77c, 77d, 77e, a maxpooling operations with a 2×2 filter is performed between each of the convolution layers. In the decoding part of the model, upsampling with a 2×2 filter is used. Furthermore, a skip connection is used between the first convolution layer 77a and the final convolution layer (which is 77i in FIG. 7), the second convolution layer 77b and penultimate convolution layer 77h (in FIG. 7, etc.), third convolution layer 77c and i−2 convolution layer 77g (where i is the number of the final convolution layer), fourth convolution layer 77d and i−3 convolution layer 77f. That is, the encoding (i.e., descending) convolutional layer is copied and concatenated to the indicated decoding (i.e., ascending) convolutional layer. Without being bound by theory, it is contemplated that these skip connections permit the feature maps of the indicated encoding part of the model to be received directly by the indicated decoding convolutional layer, which permits the indicated decoding part of the model to process extra information that might have otherwise been lost due to downsampling in the encoding part of the model. In this manner, more precise information may flow through the exemplary model, while reducing information loss or degradation.

The output of the model, which is of the same dimension as the input, is a segmentation result or a mask of the identified bones, which are the femur 105 (FIG. 8B) and the tibia 110 (FIG. 8C) in the depicted embodiment. The activation function of the last layer of the model is the binary cross entropy function, which is given by:

$$- \frac{1}{N} {\sum }_{i = 1}^{N} {y}_{i} \cdot log ( p ( {y}_{i} ) ) + ( 1 - {y}_{i} ) \cdot log ( 1 - p ( {y}_{i} ) )$$

The original input data set 10 (e.g., an input image) is used as input to the model. Femur and tibia mask images (FIGS. 8A and 8B) are used as an output of the model (see also step 7a of the exemplary method of FIG. 13). The output of the depicted model has two channels, one channel represents femur segmentation result and the other tibia segmentation result. After applying this model, the segmented femur and tibia is obtained. With the segmented masks, mathematic approaches can be used to calculate the key points of the bones. Two points confirm a line, so the identified key points can be used to obtain any of the desired alignment angles 235. It will be appreciated that in other exemplary embodiments, this model can be further configured to have further outputs, which may be used to identify the fibula 111 or other anatomical landmarks characterized by proximal pixels of high contrast.

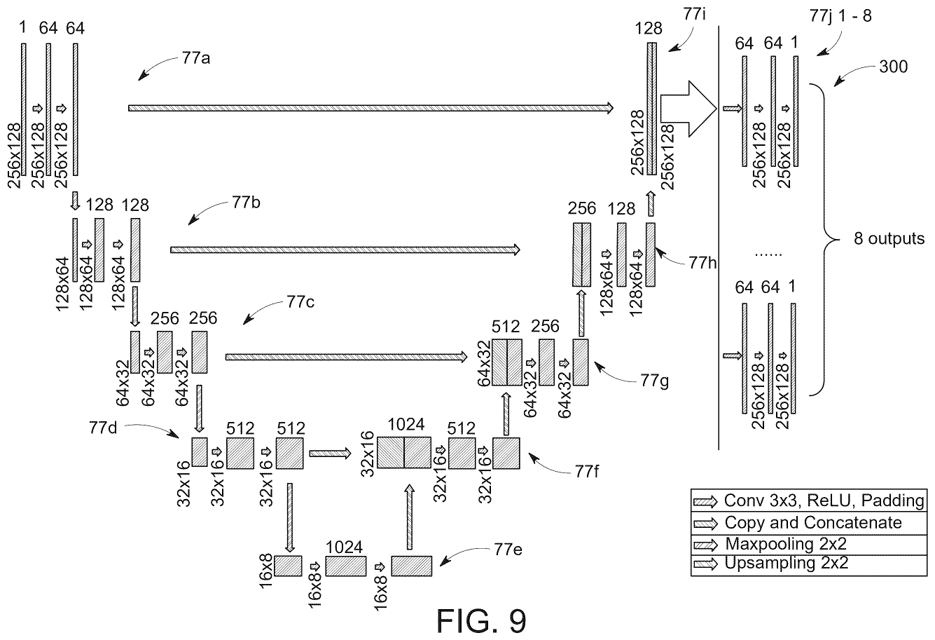

FIG. 9 is another schematic representation of an exemplary U-net CNN configured to identify elements in the target orthopedic joint 200 and further configured to identify key anatomical points on the identified or reconstructed orthopedic joint 200a, 200b for the purposes of identifying alignment angles 235 on the identified or reconstructed orthopedic joint 200a, 200b. This model is similar to the model described above with reference to FIG. 7. The encoding portion, decoding portion, and skip connections are the same as those described with reference to FIG. 7 above. However, in the present exemplary model, there are multiple outputs, each of which represents one point. In fact, the model of FIG. 9 can be seen as a replica of the model shown in FIG. 7, except that the output has a total of 16 channels (when the model is applied both to the femur and tibia) instead of 2. That is, convolutional layer 77i undergoes a further set of convolutions with a 3×3 filter, and has a further ReLU activation function applied, and has further padding eight additional times (i.e., convolutional layer 77j 1-8) to return eight outputs of the same dimensions as the initial input. When this model is trained and applied to the second target orthopedic component, an additional eight outputs are produced, thereby yielding a total of sixteen outputs that comprise the identified alignment angles 235a. Of the sixteen outputs, four outputs each can be used to identify a target joint line or axis (e.g., the distal femur joint line 52). Of the four outputs for each line, two outputs are the x-y coordinates of the line, the remaining two outputs represent a vector, written in the form of (x,y) that represents the direction of the identified line (see FIG. 10).

This key points detection model of FIG. 9 is configured to detect the key points or key anatomical features on the bones. From the key points, the lines that comprise the alignment angles 235, and by extension, the value of the alignment angles 235 themselves, can be identified and the values calculated. Two points confirm a line and the intersection of two lines confirm angles, which can include a desired identified alignment angle 235a.

For training the model, the input data set 10 to the model is the original 2D tissue-penetrating image (FIG. 8A), the outputs of the model are mask images (see FIGS. 8B and 8C) each in which target points 23 having a grey value of I have been identified. FIG. 10 shows where the target (also known as “key”) points 23 are. In order to make the target points 23 more visible, these target points 23 are exaggerated and shown larger than they actually are. After applying the deep learning model to the input data set 10, the target points 23 can be obtained. Then mathematical approaches know to those having ordinary skill in the art can be applied. Two points confirm a line, then two lines confirm an angle.

However, it is contemplated that it may be desirable to design and train the model of FIG. 9 as an independent model (i.e., not in a way that is a copy of the model of FIG. 7 with added features) because doing so could make the model more powerful for extracting key points. The activation function of the last layer of the model is the binary cross entropy function described above.

### Exemplary Embodiments Involving 3D Tissue-Penetrating Input Images

FIGS. 16A-E represent inputs, training parameters, and outputs of a further deep learning model comprising a 3D input data set 10b. FIG. 16A is a schematic representation of a tissue-penetrating input data set, wherein the input data set 10 (see FIG. 1) comprises three spatial dimensions. In this particular example, the 3D input data set 10b comes from a CT scan of a distal femur 105 in flexion. The medial distal femoral condyle 51 and the lateral distal femoral condyle 53 are likewise depicted. It will be appreciated that the same method, system, and model detailed herein can be applied to a CT scan of a proximal tibia 110 or other target bone that comprises a target orthopedic joint 200.

An advantage of starting with a 3D input data set 10b, such as one generated from a CT scan, is that the x, is that the x, y, and z spatial coordinates of the original pixels, voxels, or data points are already known by the computational machine 500. It is contemplated that this deep learning model can have the same architecture as the model described with reference to FIG. 7.

To train this model, 299 annotated knee samples (CT data sets) were used. It was found desirable to crop the multiple training data sets to have consistent dimensions throughout. Trainers manually identified target image points on the training samples from which a desired line (e.g., the distal femur joint line 52) could later be identified or drawn. The annotations are represented by the aggregated clusters 44a, 44b, and 44c of these target image points in FIG. 16B. Training masks 45a, 45b, 45c, shown in FIG. 16C were further used to train the exemplary deep learning model about the anatomical landmarks associated with each desired target image point 23. To improve precision, the black pixels inside of each training mask 45a, 45b, 45c were ignored by the model (i.e., were designated as areas where a target image point (see 44a, 44b, 44c) would not be.

FIG. 16D represents an output of this exemplary model in which the model identifies where each aggregated cluster 44a, 44b, 44c of target images points are likely to be on a new input data set 10b based on the identification of an anatomical area of the underlying bone that best corresponds to the trained masked areas 45a, 45b, 45c respectively. FIG. 16E shows the center of each of the aggregated clusters 44a, 44b, 44c being determined to yield the final target points 23a, 23b, 23c. Finally, the x, y, and z coordinates of the output of the present model are matched (or “mapped”) to the x, y, and z coordinates of the original 3D input image, thereby identifying each target point 23a, 23b, 23c relative its corresponding underlying anatomical structure in three dimensions. The desirable lines can then be determined and/or drawn connecting two or more identified target points (see 23a, 23c) and the values of the desired alignment angles 235 can thereby be calculated. In the depicted example, the distal femur joint line 52 is shown to illustrate this feature. It will be appreciated that the same model can be trained on the tibia or any other bone or bones of a desired target joint. The remaining desired lines can be displayed similarly to as shown above with reference to FIG. 10.

Determining the metes and bounds of a particular identified target orthopedic joint 200a, alignment angles 235 of the identified orthopedic joint 200a, and classifying the identified orthopedic joint 200a, into one or more classes selected from a pre-defined set of possible classes, and recommending a type of surgical procedure based on the classification of the identified target orthopedic joint 200a are considered to be within the scope of this disclosure.

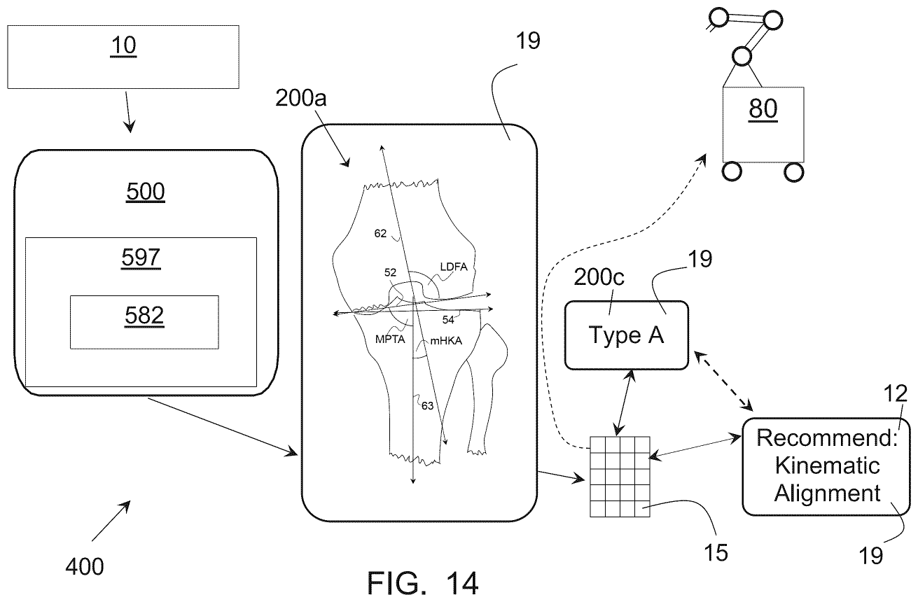

Exemplary systems may further comprise one or more databases 15 (FIG. 14). One or more databases 15 can comprise a list of types of joint alignment classification systems, a pre-defined set of possible classes for each joint alignment classification system, a group of clinically recognized surgical procedures, a list of implant sizes, a list of implant types, a list of surgical tools, a list of surgical tool subcomponents, a list of implant subcomponents, or a list of sizes for any of the foregoing elements. Combinations of the forgoing are considered to be within the scope of this disclosure.

For example, a list of types of joint alignment classification systems could include the Coronal Plane Alignment of the Knee (“CPAK”) classification system among others. If the CPAK classification system is selected as the desired classification system, the pre-defined set of possible classes for the CPAK joint alignment classification system can include a varus apex distal class, a neutral apex distal class, a valgus apex distal class, a varus neutral class, a neutral neutral class, a valgus neutral class, a varus apex proximal class, a neutral apex proximal class, and a valgus apex proximal class. Continuing with the knee example, if the exemplary systems described herein classify the identified orthopedic joint 200a as belonging to a particular class, the system may be further configured to recommend a type of surgical procedure that has been clinically shown to prolong patient comfort and implant survivorship based on the classification.

FIG. 14 represents an exemplary embodiment of an orthopedic image classification system 400 comprising: an input data set 10, the input data set 10 comprising at least one tissue-penetrating image (see 30) of a target orthopedic joint 200 and a computational machine 500. The computational machine 500 can comprise a processor 597, and memory storing instructions 582 that, when executed by the processor 597, cause the processor 597 to perform operations. These operations can include running a deep learning network 300 (see exemplary embodiments described with reference to FIGS. 4, 6, 7, and 9), wherein the deep learning network 300 is configured to identify the target orthopedic joint 200 to define an identified orthopedic joint 200a. One or more operations can further be configured to classify the identified orthopedic joint 200a into a class to define a classified joint 200c, the class being selected from a pre-defined set of possible classes stored in a database 15.

It will be appreciated that the exemplary embodiment described with reference to FIG. 14 may optionally further comprise classifying the identified orthopedic joint 200a based on identifying an alignment angle 235 of the identified orthopedic joint in any manner consistent with this disclosure to define an identified alignment angle 235a, calculating the value of said identified alignment angle 235, and comparing the value of the calculated, identified alignment angle to values of alignment angles 235 associated with each class in a pre-defined set of classes, and selecting the class that has an alignment angle value range that includes the value of the calculated alignment angle. In this manner, one or more operations can be said to be “configured to classify the identified orthopedic joint 200a into a class to define a classified joint 200c, the class being selected from a pre-defined set of possible classes.”

It will be further appreciated that the exemplary embodiment described with reference to FIG. 14 may optionally further include further operations comprising: identifying an area of bone or soft tissue loss 79 in the identified orthopedic joint 200a to define an identified loss area 79a; applying an adjustment algorithm to replace the identified loss area 79a with a reconstructed area 79b to thereby define a reconstructed orthopedic joint 200b; and identifying an alignment angle 235 of the reconstructed orthopedic joint 200b to define a reconstructed alignment angle 235b (see also FIG. 11).

In such exemplary embodiments, it will be appreciated that the operations can further comprise: classifying the reconstructed joint 200b into a class in any manner consistent with this disclosure to define a classified reconstructed joint, the class being selected from the set of pre-defined possible classes.

In certain exemplary systems image classification systems 400, the target orthopedic joint 200 is selected from a group consisting essentially of: a knee, a hip, a shoulder, an elbow, an ankle, a wrist, an intercarpal, a metatarsophalangeal, and an interphalangeal joint. In certain exemplary orthopedic image classification systems 400 one or more processors 597 are further configured to provide an output on a display 19, wherein the output is an indication or recommendation of a type of surgical procedure 12, the type of surgical procedure 12 being selected from a group of clinically recognized surgical procedures. In exemplary embodiments wherein the target orthopedic joint 200 is a knee joint, the group of clinically recognized surgical procedures can consist essentially of: a mechanical alignment procedure, an anatomic alignment procedure, and a kinematic alignment procedure. Recommending a type of clinically recognized surgical procedure based on the native constitution or alignment of a patient's pre-operative knee can be known as “functional alignment.”

In certain exemplary embodiments, the class of the identified orthopedic joint 200a is selected from the pre-defined set of possible classes, which are stored in a database 15. By way of example, these classes may comprise a varus apex distal class, a neutral apex distal class, a valgus apex distal class, a varus neutral class, a neutral neutral class, a valgus neutral class, a varus apex proximal class, a neutral apex proximal class, and a valgus apex proximal class. It will be appreciated that all classes for categorizing a target orthopedic joint 200 based on the phenotype or an anatomical feature of said target orthopedic joint 200 are considered to be within the scope of this disclosure.

It is contemplated that by using the exemplary classification systems 400 described herein, an exemplary classification system 400 can be further configured to output a recommended implant position on the identified orthopedic joint 200a. In such an embodiment, coordinates for the implant position can be provided in a coronal anatomical plane, a sagittal anatomical plane, a transverse anatomical plane, or combinations thereof. The display may further display an internal rotation or an external rotation value for the implant position.

It is further contemplated that by using the exemplary classification systems 400 in accordance with this disclosure, one or more processors 597 can be configured to further analyze contemporaneous intraoperative tracking data and gap balancing data (when the target orthopedic joint 200 is a knee joint), to recommend an implant position on the identified orthopedic joint 200a based on an analysis of contemporaneous intraoperative tracking data, the gap balancing data, and the classified joint 200c. In these exemplary embodiments describing displaying implant position, it will be understood that the positioning of trial implants, instruments, or subcomponents of implants in the manner described is considered to be within the scope of this disclosure.

Although X-ray radiographs from an X-ray imaging system may be desirable because X-ray radiographs are relatively inexpensive compared to CT scans and because the equipment for some X-ray imaging systems, such as a fluoroscopy system, are generally sufficiently compact to be used intraoperatively, nothing in this disclosure limits the use of the 2D images to X-ray radiographs unless otherwise expressly claimed, nor does anything in this disclosure limit the type of imaging system to an X-ray imaging system. Other 2D images can include by way of example: CT-images, CT-fluoroscopy images, fluoroscopy images, ultrasound images, positron emission tomography (“PET”) images, and MRI images. Other imaging systems can include by way of example: CT, CT-fluoroscopy, fluoroscopy, ultrasound, PET, and MRI systems.

Preferably, the exemplary methods can be implemented on a computer platform (e.g., a computer platform 500) having hardware such as one or more central processing units (CPU), a random access memory (RAM), and input/output (I/O) interface(s). An example of the architecture for an example computer platform 500 is provided below with reference to FIG. 5.

FIG. 5 generally depicts a block diagram of an exemplary computer platform 500 upon which one or more of the methods discussed herein may be performed in accordance with some exemplary embodiments. In certain exemplary embodiments, the computer platform 500 can operate on a single machine. In other exemplary embodiments, the computer platform 500 can comprise connected (e.g., networked) machines. Examples of networked machines that can comprise the exemplary computer platform 500 include by way of example, cloud computing configurations, distributed hosting configurations, and other computer cluster configurations. In a networked configuration, one or more machines of the computer platform 500 can operate in the capacity of a client machine, a server machine, or both a server-client machine. In exemplary embodiments, the computer platform 500 can reside on a personal computer (“PC”), a mobile telephone, a tablet PC, a web appliance, a personal digital assistant (“PDA”), a network router, a bridge, a switch, or any machine capable of executing instructions that specify actions to be undertaken by said machine or a second machine controlled by said machine.

Example machines that can comprise the exemplary computer platforms 500 can include by way of example, components, modules, or like mechanisms capable of executing logic functions. Such machines may comprise tangible entities (e.g., hardware) that is capable of carrying out specified operations while operating. As an example, the hardware may be hardwired (e.g., specifically configured) to execute a specific operation. By way of example, such hardware may have configurable execution media (e.g., circuits, transistors, logic gates, etc.) and a computer-readable medium having instructions, wherein the instructions configure the execution media to carry out a specific operation when operating. The configuring can occur via a loading mechanism or under the direction of the execution media. The execution media selectively communicate to the computer-readable medium when the machine is operating. By way of an example, when the machine is in operation, the execution media may be configured by a first set of instructions to execute a first action or set of actions at a first point in time and then reconfigured at a second point in time by a second set of instructions to execute a second action or set of actions.

The exemplary computer platform 500 may include a hardware processor 597 (e.g., a central processing unit (“CPU”), a graphics processing unit (“GPU”), a hardware processor core, or any combination thereof, a main memory 596 and a static memory 595, some or all of which may communicate with each other via an interlink (e.g., a bus) 594. The computer platform 500 may further include a display unit 19, an input device 591 (preferably an alphanumeric or character-numeric input device such as a keyboard), and a user interface (“UI”) navigation device 599 (e.g., a mouse or stylus). In an exemplary embodiment, the input device 591, display unit 19, and UI navigation device 599 may be a touch screen display. In exemplary embodiments, the display unit 19 may include holographic lenses, glasses, goggles, other eyewear, or other AR or VR display components. For example, the display unit 19 may be worn on a head of a user and may provide a heads-up-display to the user. The input device 591 may include a virtual keyboard (e.g., a keyboard displayed virtually in a virtual reality (“VR”) or an augmented reality (“AR”) setting) or other virtual input interface.

The computer platform 500 may further include a storage device (e.g., a drive unit) 592, a signal generator 589 (e.g., a speaker) a network interface device 588, and one or more sensors 587, such as a global positioning system (“GPS”) sensor, accelerometer, compass, or other sensor. The computer platform 500 may include an output controller 584, such as a serial (e.g., universal serial bus (“USB”), parallel, or other wired or wireless (e.g., infrared (“IR”) near field communication (“NFC”), radio, etc.) connection to communicate or control one or more ancillary devices.

The storage device 592 may include a machine-readable medium 583 that is non-transitory, on which is stored one or more sets of data structures or instructions 582 (e.g., software) embodying or utilized by any one or more of the functions or methods described herein. The instructions 582 may reside completely or at least partially, within the main memory 596, within static memory 595, or within the hardware processor 597 during execution thereof by the computer platform 500. By way of example, one or any combination of the hardware processor 597, the main memory 596, the static memory 595, or the storage device 592, may constitute machine-readable media.

While the machine-readable medium 583 is illustrated as a single medium, the term, “machine readable medium” may include a single medium or multiple media (e.g., a distributed or centralized database, or associated caches and servers) configured to store the one or more instructions 582.

The term “machine-readable medium” may include any medium that is capable of storing, encoding, or carrying instructions for execution by the computer platform 500 and that cause the computer platform 500 to perform any one or more of the methods of the present disclosure, or that is capable of storing, encoding, or carrying data structures used by or associated with such instructions. A non-limited example list of machine-readable media may include magnetic media, optical media, solid state memories, non-volatile memory, such as semiconductor memory devices (e.g., electronically erasable programmable read-only memory (“EEPROM”), electronically programmable read-only memory (“EPROM”), and magnetic discs, such as internal hard discs and removable discs, flash storage devices, magneto-optical discs, and CD-ROM and DVD-ROM discs.

The instructions 582 may further be transmitted or received over a communications network 581 using a transmission medium via the network interface device 588 utilizing any one of a number of transfer protocols (e.g., internet protocol (“IP”), user datagram protocol (“UDP”), frame relay, transmission control protocol (“TCP”), hypertext transfer protocol (“HTTP”), etc.). Example communication networks may include a wide area network (“WAN”), a plain old telephone (“POTS”) network, a local area network (“LAN”), a packet data network, a mobile telephone network, a wireless data network, and a peer-to-peer (“P2P”) network. By way of example, the network interface device 588 may include one or more physical jacks (e.g., Ethernet, coaxial, or phone jacks) or one or more antennas to connect to the communications network 581.

By way of example, the network interface device 588 may include a plurality of antennas to communicate wirelessly using at least one of a single-input multiple-output (“SIMO”), or a multiple-input single output (“MISO”) methods. The phrase, “transmission medium” includes any intangible medium that is capable of storing, encoding, or carrying instructions for execution by the computer platform 500, and includes analog or digital communications signals or other intangible medium to facilitate communication of such software.

Exemplary methods in accordance with this disclosure may be machine or computer-implemented at least in part. Some examples may include a computer-readable medium or machine-readable medium encoded with instructions operable to configure an electronic device to perform the exemplary methods described herein. An example implementation of such an exemplary method may include code, such as assembly language code, microcode, a higher-level language code, or other code. Such code may include computer readable instructions for performing various methods. The code may form portions of computer program products. A computer platform 500 that can execute computer readable instructions for carrying out the methods and calculations of a deep learning network can be said to be “configured to run” a deep learning network. Further, in an example, the code may be tangibly stored on or in a volatile, non-transitory, or non-volatile tangible computer-readable media, such as during execution or other times. Examples of these tangible computer-readable media may include, but are not limited to, removable optical discs (e.g., compact discs and digital video discs), hard drives, removable magnetic discs, memory cards or sticks, include removable flash storage drives, magnetic cassettes, random access memories (RAMs), read only memories (ROMS), and other media.

It is further contemplated that the exemplary methods disclosed herein may be used for preoperative planning, intraoperative planning or execution, or postoperative evaluation of the implant placement and function.

An exemplary orthopedic image processing system comprises: an input data set, the input data set comprising at least one tissue-penetrating image of a target orthopedic joint, one or more processors, and memory storing instructions that, when executed by the one or more processors, cause the one or more processors to perform operations, the operations comprising: identifying at least two bones comprising a target orthopedic joint to define an identified orthopedic joint, identifying an area of bone or soft tissue loss in the identified orthopedic joint to define an identified loss area applying an adjustment algorithm to replace the identified loss area with a reconstructed area to thereby define a reconstructed orthopedic joint, and identifying an alignment angle 235 of the reconstructed orthopedic joint to define a reconstructed alignment angle 235b.

In an exemplary orthopedic image classification system, the target orthopedic joint is selected from a group consisting essentially of: a knee, a hip, a shoulder, an elbow, an ankle, a wrist, an intercarpal, a metatarsophalangeal, and an interphalangeal joint.

In an exemplary orthopedic image classification system, the target orthopedic joint is a knee, and the knee is imaged in extension, flexion, at regular intervals from flexion to extension, or at regular intervals from extension to flexion.

An exemplary orthopedic image classification system comprises: an input data set, the input data set comprising at least one tissue-penetrating image of a target orthopedic joint, one or more processors, and non-transient memory storing instructions that, when executed by the one or more processors, cause the one or more processors to perform operations, the operations comprising: running a deep learning network, wherein the deep learning network is configured to identify the target orthopedic joint to define an identified orthopedic joint, and classifying the identified orthopedic joint into a class to define a classified joint, the class being selected from a pre-defined set of possible classes.

In an exemplary orthopedic image classification system, the operations further comprise: identifying an alignment angle of the identified orthopedic joint to define an identified alignment angle.

In an exemplary orthopedic image classification system, the operations further comprise: identifying an area of bone or soft tissue loss in the identified orthopedic joint to define an identified loss area; applying an adjustment algorithm to replace the identified loss area with a reconstructed area to thereby define a reconstructed orthopedic joint; and identifying an alignment angle of the reconstructed orthopedic joint to define a reconstructed alignment angle. In such an exemplary orthopedic image classification system, the operations may further comprise: classifying the reconstructed joint into a class to define a classified reconstructed joint, the class being selected from the set of pre-defined possible classes.

In an exemplary orthopedic image classification system, the target orthopedic joint is selected from a group consisting essentially of: a knee, a hip, a shoulder, an elbow, an ankle, a wrist, an intercarpal, a metatarsophalangeal, and an interphalangeal joint.

In an exemplary orthopedic image classification system, the operations further comprise providing a legible output on a display, wherein the legible output is an indication of a type of surgical procedure, the type of surgical procedure being selected from a group of clinically recognized surgical procedures. In such an exemplary embodiment, the target orthopedic joint is a knee joint and the group of clinically recognized surgical procedures can consist essentially of: a mechanical alignment procedure, an anatomic alignment procedure, and a kinematic alignment procedure.

In an exemplary orthopedic image classification system, the target orthopedic joint further comprises a first bone proximally disposed to a second bone, wherein the first bone is configured to be moved relative to the second bone. In one such an exemplary orthopedic image classification system, the first bone is a distal femur and the second bone is a proximal tibia. In one such exemplary system, the class is selected from the pre-defined set of possible classes consisting essentially of: a varus apex distal class, a neutral apex distal class, a valgus apex distal class, a varus neutral class, a neutral neutral class, a valgus neutral class, a varus apex proximal class, a neutral apex proximal class, and a valgus apex proximal class.

In an exemplary orthopedic image classification system, the operations further comprise providing a legible output on a display, wherein the legible output is a recommended implant position on the identified orthopedic joint. In one such exemplary embodiment, the legible output displayed on a display further comprises an implant position and coordinates in a coronal anatomical plane, sagittal anatomical plane, transverse anatomical plane, or combinations thereof.

In one such exemplary embodiment, the legible output displayed on a display further comprises an internal or external rotation of the implant position. In one such exemplary embodiment, the operations further comprise analyzing contemporaneous intraoperative tracking data and gap balancing data, displaying a legible output on a display, wherein the legible output is a recommended implant position on the identified orthopedic joint, and wherein the recommended implant position is provided based on an analysis of the contemporaneous intraoperative tracking data, the gap balancing data, and the classified joint.

In an exemplary orthopedic image classification system, the operations further comprise providing a legible output on a display, wherein the legible output is a recommended size of an implant, a surgical tool, a trial implant, or subcomponent of any of the forgoing, the recommended size being selected from a group of available pre-defined implant sizes.

In an exemplary orthopedic image classification system, the input data set comprises at least two tissue-penetrating input images of a target joint, a first input image is taken at an offset angle relative to the second input image, and the operations further comprise using photogrammetry to reconstruct a three-dimensional volume of the imaged area using image data in the first input image and the second input image. In one such exemplary embodiment, the first input image captures the target joint along an anatomical plane, and the anatomical plane is selected from the group consisting essentially of: a coronal anatomical plane, a sagittal anatomical plane, and a transverse anatomical plane.

An exemplary surgical assistance apparatus comprises: one or more processors, and non-transient memory storing instructions that, when executed by the one or more processors, cause the one or more processors to: run one or more deep learning networks, wherein the one or more deep learning networks are configured to identify a target orthopedic joint to define an identified orthopedic joint, and wherein the one or more deep learning networks are configured to classify the identified orthopedic joint into a class to define a classified joint, the class being selected from a pre-defined set of possible classes.

An exemplary orthopedic image classification system comprises: an input data set, the input data set comprising at least one tissue-penetrating image of a target orthopedic element, a computer platform configured to run a deep learning network, wherein the deep learning network is configured to identify the target orthopedic element to define an identified orthopedic element, and wherein a second deep learning network is configured to classify the identified orthopedic element into a class, the class being selected from a pre-defined set of possible classes.

In such an exemplary system, the target orthopedic element can be selected from a group consisting essentially of: a distal femur, a proximal tibia, or combinations thereof.

An exemplary knee phenotype identifying system comprises: an input data set, the input data set comprising topographical information about a distal femur and a proximal tibia of a patient's knee, a non-transient computer readable medium having instructions that, when executed by a control circuit: run one or more deep learning networks, wherein the one or more deep learning networks are configured to identify a target orthopedic joint to define an identified orthopedic joint, and wherein the one or more deep learning networks are configured to classify the identified orthopedic joint into a class to define a classified joint, the class being selected from a pre-defined set of possible classes.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. An orthopedic image processing system comprising:
an input data set, the input data set comprising at least one tissue-penetrating image of a target orthopedic joint;
one or more processors; and
memory storing instructions that, when executed by the one or more processors, cause the one or more processors to perform operations, the operations comprising:
identifying at least two bones comprising a target orthopedic joint to define an identified orthopedic joint;
identifying an area of bone or soft tissue loss in the identified orthopedic joint to define an identified loss area;
applying an adjustment algorithm to replace the identified loss area with a reconstructed area to thereby define a reconstructed orthopedic joint; and
identifying an alignment angle of the reconstructed orthopedic joint to define a reconstructed alignment angle.

2. The orthopedic image classification system of claim 1, wherein the target orthopedic joint is selected from a group consisting essentially of: a knee, a hip, a shoulder, an elbow, an ankle, a wrist, an intercarpal, a metatarsophalangeal, and an interphalangeal joint.

3. The orthopedic image classification system of claim 1, wherein the target orthopedic joint is a knee, and wherein the knee is imaged in extension, flexion, at regular intervals from flexion to extension, or at regular intervals from extension to flexion.

4. An orthopedic image classification system comprising:
an input data set, the input data set comprising at least one tissue-penetrating image of a target orthopedic joint;
one or more processors; and
memory storing instructions that, when executed by the one or more processors, cause the one or more processors to perform operations, the operations comprising:
running a deep learning network, wherein the deep learning network is configured to identify the target orthopedic joint to define an identified orthopedic joint, and
classifying the identified orthopedic joint into a class to define a classified joint, the class being selected from a pre-defined set of possible classes.

5. The orthopedic image classification system of claim 4, wherein the operations further comprise: identifying an alignment angle of the identified orthopedic joint to define an identified alignment angle.

6. The orthopedic image classification system of claim 4, wherein the operations further comprise: identifying an area of bone or soft tissue loss in the identified orthopedic joint to define an identified loss area; applying an adjustment algorithm to replace the identified loss area with a reconstructed area to thereby define a reconstructed orthopedic joint; and identifying an alignment angle of the reconstructed orthopedic joint to define a reconstructed alignment angle.

7. The orthopedic image classification system of claim 6, wherein the operations further comprise: classifying the reconstructed joint into a class to define a classified reconstructed joint, the class being selected from the set of pre-defined possible classes.

8. The orthopedic image classification system of claim 4, wherein the target orthopedic joint is selected from a group consisting essentially of: a knee, a hip, a shoulder, an elbow, an ankle, a wrist, an intercarpal, a metatarsophalangeal, and an interphalangeal joint.

9. The orthopedic image classification system of claim 4, wherein the operations further comprise providing an output on a display, wherein the output is an indication of a type of surgical procedure, the type of surgical procedure being selected from a group of clinically recognized surgical procedures.

10. The orthopedic image classification system of claim 9, wherein the target orthopedic joint is a knee joint and wherein the group of clinically recognized surgical procedures consists essentially of: a mechanical alignment procedure, an anatomic alignment procedure, and a kinematic alignment procedure.

11. The orthopedic image classification system of claim 4, wherein the target orthopedic joint further comprises a first bone proximally disposed to a second bone, and wherein the first bone is configured to be moved relative to the second bone.

12. The orthopedic image classification system of claim 11, wherein the first bone is a distal femur and the second bone is a proximal tibia.

13. The orthopedic image classification system of claim 12, wherein the class is selected from the pre-defined set of possible classes consisting essentially of: a varus apex distal class, a neutral apex distal class, a valgus apex distal class, a varus neutral class, a neutral neutral class, a valgus neutral class, a varus apex proximal class, a neutral apex proximal class, and a valgus apex proximal class.

14. The orthopedic image classification system of claim 4, wherein the operations further comprise providing an output on a display, wherein the output is a recommended implant position on the identified orthopedic joint.

15. The orthopedic image classification system of claim 14, wherein the output displayed on a display further comprises an implant position and coordinates in a coronal anatomical plane, sagittal anatomical plane, transverse anatomical plane, or combinations thereof.

16. The orthopedic image classification system of claim 14, wherein the output displayed on a display further comprises an internal or external rotation of the implant position.

17. The orthopedic image classification system of claim 14, wherein the operations further comprise analyzing contemporaneous intraoperative tracking data and gap balancing data, displaying an output on a display, wherein the output is a recommended implant position on the identified orthopedic joint, and wherein the recommended implant position is provided based on an analysis of the contemporaneous intraoperative tracking data, the gap balancing data, and the classified joint.

18. The orthopedic image classification system of claim 4, wherein the operations further comprise providing an output on a display, wherein the output is a recommended implant size, the recommended implant size being selected from a group of available pre-defined implant sizes.

19. The orthopedic image classification system of claim 4, wherein the input data set comprises at least two tissue-penetrating input images of a target joint, wherein a first input image is taken at an offset angle relative to the second input image, and wherein the operations further comprise using photogrammetry to reconstruct a three-dimensional volume of the imaged area using image data in the first input image and the second input image.

20. The orthopedic image classification system of claim 19, wherein the first input image captures the target joint along an anatomical plane, the anatomical plane selected from the group consisting essentially of: a coronal anatomical plane, a sagittal anatomical plane, and a transverse anatomical plane.
